SEQUENCE LISTING (9 sequences)

SEQ ID NO: 1 - protein, 8 aa, Mus musculus
ICPMYARV

SEQ ID NO: 2 - protein, 8 aa, Mus musculus
SIINFEKL

SEQ ID NO: 3 - protein, 489 aa, Homo sapiens
MEAPDYEVLSVREQLFHERIRECIISTLLFATLYILCHIFLTRFKKPAEFTTVDDEDATVNKIALELCTFTLAIALGAVLLLPFSIISNEVLLSLPRNYYIQWLNGSLIHGLWNLVFLFSNLSLIFLMPFAYFFTESEGFAGSRKGVLGRVYETVVMLMLLTLLVLGMVWVASAIVDKNKANRESLYDFWEYYLPYLYSCISFLGVLLLLVCTPLGLARMFSVTGKLLVKPRLLEDLEEQLYCSAFEEAALTRRICNPTSCWLPLDMELLHRQVLALQTQRVLLEKRRKASAWQRNLGYPLAMLCLLVLTGLSVLIVAIHILELLIDEAAMPRGMQGTSLGQVSFSKLGSFGAVIQVVLIFYLMVSSVVGFYSSPLFRSLRPRWHDTAMTQIIGNCVCLLVLSSALPVFSRTLGLTRFDLLGDFGRFNWLGNFYIVFLYNAAFAGLTTLCLVKTFTAAVRAELIRAFGLDRLPLPVSGFPQASRKTQHQ

SEQ ID NO: 4 - protein, 489 aa, Mus musculus
MEAADYEVLSVREQLFHDRVRECIISILLFATLYILCHIFLTRFKKPAEFTTVDDEDATVNKIALELCTFTLAVALGAVLLLPFSIISNEVLLSLPRNYYIQWLNGSLIHGLWNLVFLFSNLSLVFLMPFAYFFTESEGFAGSRKGVLGRVYETVVMLILLTLLVLGMVWVASAIVDNDKASRESLYDFWEYYLPYLYSCISFLGVLLLLVCTPLGLARMFSVTGKLLVKPRLLEDLEEQLNCSAFEEAALTRRICNPTSCWLPLDMELLHRQVLALQAQRVLLEKRRKASAWQRNLGYPLAMLCLLVLTGLSVLIVAVHILELLIDEAAMPRGMQDAALGQASFSKLGSFGAIIQVVLIFYLMVSSVVGFYSSPLFGSLRPRWHDTSMTQIIGNCVCLLVLSSALPVFSRTLGLTRFDLLGDFGRFNWLGNFYIVFLYNAAFAGLTTLCLVKTFTAAVRAELIRAFGLDRLPLPVSGFPRASRKKQHQ

SEQ ID NO: 5 - protein, 21 aa, Homo sapiens
MEAPDYEVLSVREQLFHERIR

SEQ ID NO: 6 - protein, 27 aa, Homo sapiens
SNEVLLSLPRNYYIQWLNGSLIHGLWN

SEQ ID NO: 7 - protein, 21 aa, Homo sapiens
VDKNKANRESLYDFWEYYLPY

SEQ ID NO: 8 - protein, 24 aa, Homo sapiens
DEAAMPRGMQGTSLGQVSFSKLGS

SEQ ID NO: 9 - protein, 22 aa, Homo sapiens
SRTLGLTRFDLLGDFGRFNWLG

(19) United States
(12) Patent Application Publication
(10) Pub. No.: US 2021/0269519 A1
(43) Pub. Date: Sep. 2, 2021

(54) Methods and Compositions for Regulating an Immune Response

(71) Applicant: Board of Regents of the University of Texas System, Austin, TX (US)

(72) Inventors: Bruce Beutler, Austin, TX (US); Jin Huk Choi, Austin, TX (US); Xue Zhong, Austin, TX (US); Evan Nair-Gill, Austin, TX (US)

(21) Appl. No.: 17/253,963

(22) PCT Filed: Jun. 26, 2019
(86) PCT No.: PCT/US2019/039343

§ 371 Date: Dec. 18, 2020

Related U.S. Application Data
(60) Provisional application No. 62/689,907, filed on Jun. 26, 2018.

(51) Int. Cl. C07K 16/28; A61K 48/00; A61P 37/04; A61P 37/06; A61K 38/17; G01N 33/68

(52) U.S. Cl. CPC C07K 16/28; A61K 48/005; A61P 37/04; A61P 37/06; G01N 2800/245; G01N 33/6893; G01N 2800/7095; G01N 2800/24; A61K 38/1796

(57) ABSTRACT

The present disclosure generally relates to a method for providing an immune suppressive therapy, in particular by inhibiting LMBR1L (limb region 1 like) in a subject in need thereof. Also, provided herein are compositions and kits that can be used in such methods.

### CROSS-REFERENCE TO RELATED APPLICATIONS

This application claims priority to and the benefit of U.S. Provisional Patent Application No. 62/689,907 filed Jun. 26, 2018, incorporated herein by reference in its entirety.

### FIELD

The present disclosure generally relates to a method for providing an immune suppressive therapy, in particular by inhibiting limb region 1 like (LMBR1L) in a subject in need thereof. Also, provided herein are compositions and kits that can be used in such methods.

### BACKGROUND

Diseases associated with an excessive or overactive immune system, such as inflammatory and autoimmune diseases, are among the most prevalent diseases in the United States, affecting more than 23.5 million people. Some inflammatory and autoimmune diseases are life-threatening, and most are debilitating and require a lifetime of treatment. Despite multiple therapeutic approaches, the proportion of the population living with an inflammatory or autoimmune related disease is predicted to increase by at least 37% before 2030.

Excessive inflammation caused by abnormal recognition of host tissue as foreign, or prolongation of the inflammatory process, may lead to autoimmune or inflammatory diseases as diverse as asthma, diabetes, arteriosclerosis, cataracts, reperfusion injury, and cancer, to post-infectious syndromes such as in infectious meningitis, and to rheumatic diseases such as systemic lupus erythematosus and rheumatoid arthritis. The centrality of the immune response in these varied diseases makes regulation of the immune system a critical component of disease treatment. Although an abnormal inflammatory response may be modulated by anti-inflammatory agents such as corticosteroids, immunosuppressants, non-steroidal anti-inflammatory drugs (NSAID), COX-2 inhibitors, and protease inhibitors, many of these agents have significant side effects. For example, corticosteroids may induce Cushingoid features, skin thinning, increased susceptibility to infection, and suppression of the hypothalamic-pituitary-adrenal axis. Also, since inflammatory and autoimmune diseases are often chronic, they generally require lifelong treatment and monitoring. Thus, a need exists for effective methods and compositions to treat inflammatory and autoimmune diseases.

### SUMMARY

Disclosed herein are methods of providing an immune suppressive therapy to treat a disease (e.g., an inflammatory disease, autoimmune disease, graft versus host disease, or an allograft rejection) in a subject in need thereof, the method comprising inhibiting limb region 1 like (LMBR1L) in the subject. The methods include suppressing or reducing an immune response in a subject in need thereof, as well as methods related to decreasing the level of T cells, B cells, NK and/or NK T cells in a subject in need thereof. Also, provided herein are compositions and kits that can be used in such methods.

In one aspect, a method of providing an immune suppressive therapy is provided, which comprises inhibiting limb region 1 like (LMBR1L) in a subject in need thereof, thereby suppressing an immune response.

In some embodiments, said inhibiting comprises reducing the number of common lymphoid progenitors and/or lymphocytes in the subject. The lymphocytes can include, for example, one or more of T cells, B cells, NK and NK T cells.

The subject can have an inflammatory disease, autoimmune disease, graft versus host disease, or an allograft rejection. In some embodiments, the autoimmune disease can be systematic lupus erythematosus (SLE), Hashimoto's thyroiditis, Grave's disease, type I diabetes, multiple sclerosis and/or rheumatoid arthritis.

In various embodiments, the method can further include administering to the subject an effective amount of an LMBR1L inhibitor such as an anti-LMBR1L antibody or antigen binding fragment thereof, which binds to LMBR1L, such as an extracellular domain of LMBR1L.

Another aspect relates to an LMBR1L inhibitor such as anti-LMBR1L antibody or antigen binding fragment thereof, which binds to limb region 1 like (LMBR1L), preferably an extracellular domain of LMBR1L.

A further aspect relates to a pharmaceutical composition for immunosuppression, comprising the LMBR1L inhibitor such as antibody or antigen binding fragment thereof disclosed herein, and a pharmaceutically acceptable carrier.

Also disclosed herein is use of LMBR1L inhibitors such as anti-LMBR1L antibody or antigen binding fragment thereof disclosed herein, for the manufacture of a medicament for suppressing or reducing an immune response.

### BRIEF DESCRIPTION OF THE DRAWINGS

FIGS. 1A-1R. A heritable lymphopenia caused by LMBR1L deficiency in mice. (A) Manhattan plot. −Log₁₀ P-values plotted vs. the chromosomal positions of mutations identified in the affected pedigree. (insets) Representative flow cytometric plot of B220⁺ and CD3⁺ peripheral blood lymphocytes in wild-type (WT) and strawberry mice. (B) LMBR1L topology. The schematic shows the location of the Lmbr1l point mutation, which results in substitution of cysteine 212 for a premature stop codon (C212*) in the LMBR1L protein. (C-J, M, N) Frequency and surface marker expression of T (C-F), B (H-J), NK (M), and NK1.1⁺ T (N) cells in the peripheral blood from 12-week-old Lmbr1l^{-/-} or Cers5^{-/-} mice generated by the CRISPR/Cas9 system. (K) T cell-dependent β-gal-specific antibodies 14 days after immunization of 12-week-old Lmbr1l^{-/-} or Cers5^{-/-} mice with a recombinant SFV vector encoding the model antigen, β-gal (rSFV-βGal). Data presented as absorbance at 450 nm. (L) T cell-independent NP-specific antibodies 6 days after immunization of 13-week-old Lmbr1l^{-/-} or Cers5^{-/-} mice with NP-Ficoll. Data presented as absorbance at 450 nm. (0) Quantitative analysis of the β-gal-specific cytotoxic T cell killing response in Lmbr1l^{-/-} mice that were immunized with rSFV-βGal. An equal mixture of ICPMYARV (SEQ ID NO. 1) peptide 03-gal-specific MHC I epitope for mice with H-2d haplotype) pulsed CFSE^{hi} and unpulsed CFSE^{lo} splenocytes were adoptively transferred to immunized mice by retro-orbital injection. Mice were bled 48 h following adoptive transfer and killing of CFSE-labeled target cells was analyzed by flow cytometry. (P) Lmbr1l^{-/-} mice generate reduced antigen-specific CD8⁺ T cell responses to aluminum hydroxide precipitated ovalbumin (OVA/alum). Lmbr1l^{-/-} and wild-type littermates were immunized with OVA/alum at day 0. Total and memory K^{b}/SIINFEKL (SEQ ID NO. 2) tetramer-positive CD8⁺ T cells were analyzed at day 14 by flow cytometry using CD44 and CD62L surface markers. (Q) NK cell cytotoxicity against MHC class I-deficient (B2 m^{-/-}) target cells in Lmbr1l^{-/-} mice. An equal mixture of CellTrace Violet-labeled C57BL/6J (Violet^{lo}) and B2 m^{-/-} (Violet^{hi}) cells were transferred into recipient mice and NK cell cytotoxicity toward target cells was analyzed by flow cytometry 48 h after injection. (R) Viral DNA copies in livers from Lmbr1l^{-/-} mice 5 days after infection with 1.5×10⁵ pfu MCMV Smith strain. Each symbol represents an individual mouse (C-R). P-values were determined by one-way ANOVA with Dunnett's multiple comparisons (C-O, Q, R) or Student's t-test (P). Data are representative of two independent experiments (C-J, M, N) or one experiment (K, L, O-R) with 5-24 mice per genotype. Error bars indicate S.D. *P<0.05; ***P<0.001.

FIGS. 2A-2M. A cell-intrinsic failure of lymphocyte development. (A-D) Repopulation of lymphocytes in spleen (A, B), thymus (C), and bone marrow (D) 12 weeks after reconstitution of irradiated wild-type (C57BL/6J; CD45.1) and strawberry (CD45.2) recipients with strawberry (CD45.2) or wild-type (C57BL/6J; CD45.1) bone marrow, or Rag2^{-/-} recipients with a 1:1 mixture of Lmbr1l^{st/st} (CD45.2) and wild-type (C57BL/6J; CD45.1) bone marrow. Representative flow cytometric scatter plots of B and T cells (A), NK cells (B), thymocytes (C), and bone marrow B cells. MR: mature recirculating B cells, Trans.: transitional B cells, Imm.: immature B cells. Numbers adjacent to outlined areas or in quadrants (A-D) indicate percent cells in each. (A, B, E-G) Reconstitution of B (A, E), T (A, F), and NK (B, G) cells in the spleen of recipients with donor-derived cells 12 weeks after engraftment. (C, D, H-K) Repopulation of donor-derived T cell subsets in thymus (C, H, I) and B cell subsets in bone marrow (D, J, K) in recipients rescued from lethal irradiation. (L, M) The frequencies (L) and total numbers (M) of stem and progenitor cell subsets per femur in the LSK⁺ and LK⁺ compartments in the bone marrow of Lmbr1l^{-/-} and wild-type littermates as determined by flow cytometry. Each symbol represents an individual mouse (E-M). P-values were determined by one-way ANOVA with Dunnett's multiple comparisons (E-K) or Student's t-test (L, M). Data are representative of two independent experiments with 6-7 mice per genotype. Error bars indicate S.D. *P<0.05; **P<0.01; ***P<0.001.

FIGS. 3A-3M. LMBR1L-deficient T cells die in response to expansion signals. (A) LMBR1L-deficient peripheral T cells are activated. Flow cytometric analysis of CD44 expression on T cells in the thymi and spleens of 12-week-old Lmbr1l^{-/-} and wild-type littermates. (B) Immunoblot analysis of TCF1/7, LEF1, Akt, phospho-Akt, S6, phospho-S6, phospho-p70S6K, phospho-p44/p42 MAPK, and GAPDH in total cell lysates (TCLs) of pooled CD8⁺ T cells from Lmbr1l^{-/-} or wild-type littermates. (C) Annexin V staining of CD4⁺ or CD8⁺ T cells in peripheral blood obtained from 14-week-old wild-type or strawberry mice. (D) IL-7Rα expression on CD3⁺ T cells in peripheral blood obtained from 12-week-old Lmbr1l^{-/-} and wild-type littermates. (E-G) Impaired antigen-specific expansion of LMBR1L-deficient T cells. A 1:1 mixture of CellTrace Violet-labeled Lmbr1l^{-/-} (CD45.2) and Far Red-stained wild-type OT-I T cells (CD45.2) was adoptively transferred into wild-type hosts (C57BL/6J; CD45.1). Representative flow cytometric scatter plots (E) and histograms (F), and quantification of total numbers (G) of CellTrace Violet- or Far Red-positive wild-type or Lmbr1l^{-/-} OT-I T cells harvested from the spleens of wild-type (C57BL/6J; CD45.1) hosts, 48 or 72 h after immunization with soluble OVA or sterile PBS (vehicle) as a control. (H-M) Impaired homeostatic expansion of LMBR1L-deficient T cells. An equal mixture of CellTrace Violet-labeled or CellTrace Far Red-stained pan T cells isolated from the spleen (H-J) or mature single-positive thymocytes (K-M) from Lmbr1l^{-/-} or wild-type littermates were adoptively transferred into sublethally irradiated (8.5 Gy) wild-type hosts (C57BL/6J; CD45.1). Representative flow cytometric scatter plots (H, K) and histograms (I, L), and quantification of total numbers (J, M) of CellTrace Violet- or CellTrace Far Red-positive cells harvested from the spleens of sublethally irradiated or unirradiated wild-type hosts, 4 or 7 days after transfer. Numbers adjacent to outlined areas indicate percent cells in each ±SD. Each symbol represents an individual mouse (A, C, D, G, J, M). P-values were determined by Student's t-test (A, C, D) or one-way ANOVA with Dunnett's multiple comparisons (G, J, M). Data are representative of two independent experiments with 4-29 mice per genotype or group. Error bars indicate S.D. *P<0.05; **P<0.01; ***P<0.001.

FIGS. 4A-4C. LMBR1L negatively regulates Wnt signaling. (A, B) LMBR1L physically interacts with components of the Wnt signaling pathway. (A) Human protein microarray revealed binding between LMBR1L and GSK-3β proteins. A construct expressing both N-terminus FLAG-tagged and C-terminus V5-tagged human LMBR1L was transfected into HEK293T cells and the recombinant protein was purified using anti-FLAG M2 agarose beads. Binding between recombinant human LMBR1L and purified human proteins printed in duplicate on the microarray slide was probed with anti-V5-Alexa 647 antibody. (B) HEK293T cells were transfected with either FLAG-tagged GSK-3β, β-catenin, ZNRF3, RNF43, FZD6, LRP6, DVL2, or empty vector (EV) and HA-tagged LMBR1L. Lysates were subsequently immunoprecipitated using anti-FLAG M2 agarose and immunoblotted with antibodies against HA or FLAG. (C) Immunoblot analysis of β-catenin, phospho-β-catenin, AXIN1, DVL2, GSK-3α/β, phospho-GSK-3β, CK1, β-TrCP, c-Myc, p53, p21, caspase-3, cleaved caspase-3, caspase-9, cleaved caspase-9, and GAPDH in TCLs of pooled CD8⁺ T cells from Lmbr1l^{-/-} or wild-type littermates. Data are representative of three-to-five independent experiments.

FIGS. 5A-5G. LMBR1LGP78UBAC2 complex regulates maturation of Wnt receptors within the ER. (A) Immunoblots of the indicated proteins in membrane and TCLs of pooled CD8⁺ T cells isolated from the spleens of 12-week-old Lmbr1l^{-/-} or wild-type littermates. The upper band of FZD6 or LRP6 (red arrowhead) is the mature form; the lower band (blue arrowhead) is the ER form of FZD6 or LRP6 (also applies to B, C, E, F). Expression of GRP94 or BiP was determined with a KDEL antibody. GAPDH was used as loading control. *, an unknown KDEL-positive protein whose expression is unchanged. (B) HEK293T cells were transfected with FLAG-tagged FZD6 and either HA-tagged LMBR1L, UBAC2, GP78, or empty vector. TCLs were immunoprecipitated using anti-FLAG M2 agarose beads and immunoblotted with antibodies against FLAG, HA, and Ubiquitin (UB). GAPDH was used as a loading control. (C) HEK293T cells were transfected with FLAG-tagged LRP6 and HA-tagged LMBR1L, UBAC2, or empty vector. TCLs were immunoblotted using the indicated antibodies. (D) ER or plasma membrane proteins were isolated from LMBR1L-FLAG knock-in (KI) or parental HEK293T cells (WT). Endogenous LMBR1L expression was then analyzed by immunoblotting using a FLAG antibody. Expression of calnexin, E-cadherin, or α-tubulin were used as loading controls for ER, plasma membrane, or cytosol, respectively. (E) Immunoblots of indicated proteins in TCLs of pooled CD8⁺ T cells isolated from the spleens of 6-week-old Gp78^{-/-} or wild-type mice. (F) Constructs encoding FLAG-tagged LRP6 and HA-tagged LMBR1L were transfected into Gp78^{-/-} or parental HEK293T cells. TCLs were immunoblotted using the indicated antibodies. (G) HEK293T cells were transfected with FLAG-tagged β-catenin and either HA-tagged LMBR1L, UBAC2, GP78, or empty vector. TCLs were immunoprecipitated using anti-FLAG M2 agarose beads and immunoblotted with antibodies against FLAG, HA, and Ubiquitin (UB). GAPDH was used as a loading control. Data are representative of two-to-five independent experiments.

FIGS. 6A-6B. LMBR1L stabilizes GSK-3β. (A) HEK293T cells were transfected with FLAG-tagged GSK-3β and either HA-tagged LMBR1L or empty vector. TCLs were immunoprecipitated using anti-FLAG M2 agarose beads and immunoblotted with antibodies against p-GSK-3β, FLAG, and HA. GAPDH was used as a loading control. (B) HEK293T cells were transfected with FLAG-tagged GSK-3β and either HA-tagged LMBR1L or empty vector. The cells were treated with cyclohexamide (CHX) 14 h after transfection and harvested at various times post-treatment. TCLs were immunoblotted with the indicated antibodies. Two primary antibodies (anti-HA and GAPDH) were co-incubated to visualize LMBR1L (red arrowhead) and GAPDH (blue arrowhead, a loading control) on one membrane. Data are representative of three independent experiments.

FIGS. 7A-7C. Deletion of β-catenin (Ctnnb1) attenuates apoptosis caused by LMBR1L-deficiency. (A-C) Growth curve (A) and Annexin V/PI staining (B) of Lmbr1l^{-/-}, Ctnnb1^{-/-}, and Lmbr1l^{-/-}; Ctnnb1^{-/-} EL4 cells generated by the CRISPR/Cas9 system (n=3-5 clones/genotype), and parental wild-type (WT) EL4 cells. Numbers adjacent to outlined areas (B) indicate percent cells in each. (C) Quantification of the percentage of viable, apoptotic, and necrotic Lmbr1l^{-/-}, Lmbr1l^{-/-}; Ctnnb1^{-/-}, or parental WT EL4 cells. Each symbol represents an individual cell clone. P-values were determined by one-way ANOVA with Dunnett's multiple comparisons. Data are representative of three independent experiments. Error bars indicate S.D. *P<0.05; **P<0.01.

FIGS. 8A-8C. Identification of a mutation in Lmbr1l as causative for severe lymphopenia in mice. To distinguish the effects of mutations in Cers5 versus Lmbr1l, third-generation (G3) descendants of a single ENU-mutagenized male mouse heterozygous for the mutations in Cers5 and Lmbr1l were intercrossed to segregate the two ENU-induced point mutations. Peripheral blood lymphocytes from offspring with the indicated genotypes were analyzed by flow cytometry. (A) Representative flow cytometric analysis of CD3⁺ and B220⁺ cells in the peripheral blood of 12-week-old mice. (B) Activation marker (CD44 and CD62L) expression on the surface of CD3⁺CD8⁺ T cells in the peripheral blood. (C) Quantification of the frequency in peripheral blood of B and T cells, and the frequency of naïve, central memory (CM), and effector memory (EM) CD8⁺ T cells based on CD62L and CD44 expression. Each symbol represents an individual mouse. P-values were determined by one-way ANOVA with Dunnett's multiple comparisons. Data are representative of three independent experiments with 4-9 mice per genotype. Error bars indicate S.D. *P<0.05; ***P<0.001.

FIGS. 9A-9H. Whole-blood leukocyte counts in 12-week-old Lmbr1l^{-/-} and wild-type littermates. Each symbol represents an individual mouse. P-values were determined by Student's t-test. Data are representative of three independent experiments with 12-14 mice per genotype. Error bars indicate S.D. *P<0.05; ***P<0.001; ns, not significant with P>0.05.

FIGS. 10A-10N. Summary of the phenotypes observed in mice carrying an ENU-induced Lmbr1l mutation. (A-H, K, L) Frequency and surface marker expression of T (A-D), B (F-H), NK (K), and NK T (L) cells in the peripheral blood from wild-type C57BL/6J (WT) mice, or a pedigree of G3 descendants of a single ENU-mutagenized male mouse with REF (+/+), HET (strawberry/+), or VAR (strawberry/strawberry) genotypes for Lmbr1l. (I, J) T cell-dependent (I) or T cell-independent (J) antibody responses in G3 mice with the indicated genotypes for Lmbr1l following immunization with rSFV-βGal or NP-Ficoll, respectively. Data presented as absorbance at 450 nm. (M, N) In vivo cytotoxic T lymphocyte activity against target cells pulsed with β-Gal, a model antigen encoded in rSFV-βGal (M), or NK cell cytotoxicity against MHC class I-deficient (B2 m^{-/-}) target cells (N) in G3 mice with indicated genotypes for Lmbr1l 48 h after adoptive transfer. Each symbol represents an individual mouse. The significance of differences between genotypes was determined by one-way ANOVA with Dunnett's multiple comparisons. Data are representative of three independent experiments (A-L) or one experiment (M, N) with 6-22 mice per genotype. Error bars indicate S.D. ***P<0.001.

FIGS. 11A-11O. Flow cytometric quantification of developing thymocytes and immune cells in the spleens of Lmbr1l^{-/-} and wild-type littermates. (A-E) Thymocytes were analyzed by flow cytometry for CD4, CD8, CD25, and CD44 surface markers. (F-O) Splenocytes were analyzed by flow cytometry for surface markers encompassing the major immune lineages: B220, CD3ε, CD4, CD5, CD8α, CD11b, CD11c, CD19, CD43, F4/80, and NK1.1. Each symbol represents an individual mouse. P-values were determined by Student's t-test. Data are representative of two independent experiments with 8 mice per genotype. Error bars indicate S.D. *P<0.05; **P<0.01; ***P<0.001; NS, not significant with P>0.05.

FIGS. 12A-12C. Reduced antigen-specific CD8⁺ T cell responses in Lmbr1l^{-/-} mice. Lmbr1l^{-/-}, wild-type littermates, and OT-I mice were immunized with aluminum hydroxide precipitated ovalbumin (OVA/alum) at day 0. Frequency of total (A, C) and memory (B, C) K^{b}/SIINFEKL tetramer-positive CD8⁺ T cells were analyzed at day 14 by flow cytometry using CD44 and CD62L surface markers. CM, central memory; EM, effector memory. Each symbol represents an individual mouse (C, n=4/genotype). P-values were determined by Student's t-test. Data are representative of two independent experiments. Error bars indicate S.D. ***P<0.001; NS, not significant with P>0.05.

FIGS. 13A-13J. Expression profile of Lmbr1l and normal cytokine secretion by Lmbr1l^{-/-} peritoneal macrophages (PMs) in response to stimulation. (A-B) Lmbr1l transcript levels normalized to Gapdh mRNA in different tissues (A) and immune cells (B) of C57BL/6J mice at 8 wks of age (n=12). HSPC: hematopoietic stem/progenitor cells. (C-J) PMs from Lmbr1l^{-/-} and wild-type littermates were stimulated with Pam3CSK4 (TLR2/1 ligand; C), poly(I:C) (TLR3 ligand; D), lipopolysaccharide (LPS; TLR4 ligand; E), R848 (TLR7 ligand; F), CpG-oligodeoxynucleotide (CpG-ODN; TLR9 ligand; G), dsDNA (H), nigericin (inflammasome; I), and flagellin (TLR5 ligand; J) in vitro at the concentrations indicated in the materials and methods. IFN-α, IL-1β, and TNF-α in the culture medium were measured by ELISA 4 h later. Each symbol represents an individual mouse. P-values were determined by Student's t-test. Data are representative of two independent experiments with 4-6 mice per genotype. Error bars indicate S.D. NS, not significant with P>0.05.

FIGS. 14A-14B. Lmbr1l^{-/-}-derived hematopoietic stem cells have a disadvantage in repopulating lymphoid-primed multipotent progenitors (LMPP) and common lymphoid progenitors (CLP) in competitive bone marrow chimeras. (A-B) Repopulation of hematopoietic stem cell and progenitor populations in competitive bone marrow chimeras. (A) A 1:1 mixture of Lmbr1l^{+/+} BM (CD45.2) and congenic WT BM (C57BL/6J; CD45.1) competitor cells were injected into lethally-irradiated Rag2^{-/-} recipients. (B) A 1:1 mixture of Lmbr1l^{-/-} BM (CD45.2) and congenic WT BM (C57BL/6J; CD45.1) competitor cells were injected into lethally irradiated Rag2^{-/-} recipients. Donor chimerism levels in the peripheral blood was assessed using congenic CD45 markers at 8 weeks post-transplant. Each symbol represents an individual mouse (n=4-6/group). P-values were determined by Student's t-test. Error bars indicate S.D. **P<0.01; ***P<0.001.

FIGS. 15A-15C. Apoptosis of Lmbr1l^{-/-} or Lmbr1l^{st/st} CD8⁺ T cells in response to antigen-specific or homeostatic expansion signals. (A) Annexin V staining of adoptively transferred wild-type OT-I or Lmbr1l^{-/-} OT-I T cells isolated from the spleens of wild-type (C57BL/6J; CD45.1) recipient mice, 48 h after injection of soluble OVA. (B) Annexin V staining of adoptively-transferred wild-type or Lmbr1l^{st/st} CD3⁺ T cells isolated from spleens of sub-lethally irradiated (8.5 Gy) wild-type hosts (C57BL/6J; CD45.1) 4 days after transfer. (C) Annexin V staining of adoptively-transferred wild-type or Lmbr1l^{-/-} mature single positive (SP) thymocytes isolated from spleens of sublethally irradiated (8.5 Gy) wild-type hosts (C57BL/6J; CD45.1) 4 days after transfer. Each symbol represents an individual mouse. P-values were determined by Student's t-test. Data are representative of two independent experiments with 6 mice per genotype. Error bars indicate S.D. **P<0.01; ***P<0.001.

FIGS. 16A-16B. Lmbr1l^{-/-} CD4⁺ and CD8⁺ T cells can home to secondary lymphoid organs, but have proliferative defects in response to homeostatic expansion signals. (A) A 10:1 mixture of CellTrace Violet-labeled (Lmbr1l^{-/-}) or CellTrace Far Red-labeled (Lmbr1l^{+/+}) pan T cells isolated from the spleen were adoptively transferred into sublethally irradiated (8.5 Gy) wild-type hosts (C57BL/6J; CD45.1). Representative flow cytometric scatter plots (A) and histograms (B) of CellTrace Violet or CellTrace Far Red dilutions in cells harvested from spleens of sublethally irradiated or unirradiated wild-type hosts 7 days after transfer. Data are representative of two independent experiments with 5-7 mice per group.

FIGS. 17A-17E. Enhanced intrinsic and extrinsic caspase activation in Lmbr1l^{st/st} T cells in response to stimulation. (A, B) Immunoblot analysis of caspase processing or cleavage of PARP in lysates of pooled splenic CD8⁺ T cells from Lmbr1^{st/st} or WT littermates upon TNF-α (10 ng/ml; A) or FasL (25 ng/ml; B) stimulation for 0.5, 1, 2, 4 h or left untreated. (C, D, E) Representative flow cytometric analysis of CD3⁺ and B220⁺ cells in peripheral blood of 12-week-old Lmbr1l^{-/-}; Tnf^{-/-} (C), Lmbr1l^{-/-}; Fas^{lpr/lpr} (D), Lmbr1l^{-/-}; Casp3^{-/-} (E), or littermates with the indicated genotypes. Data are representative of three independent experiments with 3-7 mice per genotype.

FIGS. 18A-18C. LCN3 deficiency has no effect on lymphocyte development in mice. LMBR1L has been identified as a receptor for human lipocalin-1 (13, 14). To determine if lipocalin plays an important role in lymphopoiesis, we generated mice deficient for LCN3, the mouse orthologue of human lipocalin-1, using the CRISPR/Cas9 system. (A) Representative flow cytometric analysis of CD3⁺ and B220⁺ cells in the peripheral blood of 12-week-old Lcn3^{-/-} or WT littermates. (B) Activation marker (CD44 and CD62L) expression on the surface of CD3⁺CD8⁺ T cells in the peripheral blood. (C) Quantification of the frequency in peripheral blood of B and T cells, and the frequency of naïve, central memory (CM), and effector memory (EM) CD8⁺ T cells based on CD44 and CD62L expression. Each symbol represents an individual mouse. P-values were determined by Student's t-test. Data are representative of two independent experiments with 4-7 mice per genotype. Error bars indicate S.D. NS, not significant with P>0.05.

FIGS. 19A-19D. LMBR1L physically interacts with components of the endoplasmic reticulum-associated degradation (ERAD) system. (A, B) Constructs expressing either FLAG-tagged UBAC2, UBXD8, VCP, or empty vector were expressed with HA-tagged LMBR1L or UBAC2 in HEK293T cells. Cell lysates were subsequently immunoprecipitated using anti-FLAG M2 agarose and immunoblotted with antibodies against HA or FLAG. (C, D) Constructs expressing either the FLAG-tagged GP78 or empty vector were expressed with HA-tagged LMBR1L or UBAC2 in HEK293T cells. Immunoprecipitation and immunoblot were performed as described in (A, B). Data are representative of two independent experiments.

FIGS. 20A-20C. LMBR1L deficiency results in nuclear accumulation of β-catenin. (A) Intracellular β-catenin in thymocyte subsets from Lmbr1l^{-/-} or wild-type littermates. (B) Immunoblot analysis of β-catenin in total cell lysates of pooled mature single positive thymocytes, naïve pan T cells, and pan T cells from the spleens of Lmbr1l^{-/-} and WT littermates. (C) Immunoblot analysis of β-catenin in total cell lysates as well as cytosolic and nuclear extracts of pooled CD8⁺ T cells from Lmbr1l^{-/-} or WT littermates. GAPDH and Histone H3 were used as markers for purity of cytosolic and nuclear fractions. Each symbol represents an individual mouse. P-values were determined by Student's t-test. Data are representative of two independent experiments with 6 mice per genotype. Error bars indicate S.D. **P<0.01; ***P<0.001.

FIGS. 21A-21B. Immunoblot analysis of Wnt components in CD4⁺ T and B cells. Immunoblot analysis of LRP6, phospho-LRP6, β-catenin, phospho-β-catenin, AXIN1, DVL2, CK1, β-TrCP, c-Myc, caspase-3, cleaved caspase-3, caspase-9, cleaved caspase-9, and GAPDH in total cell lysate of pooled CD4⁺ T (A) or pan B (B) cells from Lmbr1l^{-/-} or WT littermates.

FIGS. 22A-22E. Normal proliferation and β-catenin activation in the Lmbr1l^{-/-} small intestine and colon. (A, B) Wild-type and Lmbr1l^{-/-} intestines were stained for β-catenin (A) or Ki-67 (B). Scale bars: 50 n=3-4 mice/genotype; representative images are shown. (C) Ten fields of view per mouse were averaged to obtain the β-catenin mean fluorescence intensity (MFI) value. (D) Quantification of proliferating cells per crypt (Ki-67⁺) in Lmbr1l^{-/-} and wild-type littermates. (E) Percentage of initial body weight on day 10 of 1.5% DSS treatment in drinking water for wild-type C57BL/6J mice (WT) or a pedigree of G3 descendants of a single ENU-mutagenized male mouse with REF (+/+), HET (strawberry/+), or VAR (strawberry/strawberry) genotypes for Lmbr1l. Each symbol represents an individual mouse. n=6-22 mice/genotype. P-values were determined by Student's t-test (C, D) or one-way ANOVA with Dunnett's multiple comparisons (E). Error bars indicate S.D. NS, not significant with P>0.05.

FIG. 23. LMBR1L induces retention of Frizzled-6 (FZD6) in the ER and inhibits its expression on the cell surface. Confocal fluorescent live cell images of HEK293T cells cultured in glass bottom 8-well glass chambers. Cells were transfected with FZD6-GFP together with empty vector (upper panel) or LMBR1L-HA (bottom panel). The ER was visualized by infecting transfected cells with CellLight ER-RFP baculovirus (RFP-KDEL) 16 h before images were captured. Blue arrows indicate FZD-GFP expression on plasma membrane. Scale bars: 10 μm. Images are representative of three independent experiments, each with duplicate wells per transfection/baculovirus infection condition. Three fields of view were captured from each well per experiment.

FIGS. 24A-24B. Expression of Wnt components in resting Ubac2^{-/-} or Gp78^{-/-} cells. (A) Immunoblot analysis of FZD6, LRP6, β-catenin, UBAC2, GP78, GAPDH, and α-tubulin in total cell lysates of parental WT, Ubac2^{-/-}, or Gp78^{-/-} HEK293T (A) or EL4 cells (B). Red arrowheads indicate the mature form, and the blue arrowheads indicate the immature form. Data are representative of three independent experiments.

FIG. 25. Effect of UBAC2 on LMBR1L-mediated FZD6 maturation. Constructs encoding the FLAG-tagged FZD6 and EGFP were co-expressed with increasing amounts of HA-tagged LMBR1L in parental HEK293T and Ubac2^{-/-} cells. Total cell lysates were immunoblotted using the indicated antibodies. Red arrowhead indicates the mature form and blue arrowhead indicates the ER form of FZD6. Data are representative of three independent experiments.

FIGS. 26A-26B. GP78 physically interacts with β-catenin. (A) FLAG-tagged β-catenin constructs were expressed with either HA-tagged GP78 or empty-HA vector in HEK293T cells. Cell lysates were subsequently immunoprecipitated using anti-FLAG M2 agarose and immunoblotted with antibodies against HA or FLAG. Data are representative of two independent experiments. (B) A construct encoding FLAG-tagged β-catenin was transfected in Gp78^{-/-} or parental HEK293T cells. Total cell lysates were immunoblotted using the indicated antibodies.

FIG. 27. Effect of LMBR1L on expression of destruction complex proteins. HEK293T cells were co-transfected with constructs encoding the FLAG-tagged Axin1, DVL2, or GSK-3β and HA-tagged LMBR1L or empty vector. Total cell lysates were immunoblotted using the indicated antibodies.

FIG. 28. Model of LMBR1L function in lymphopoiesis. LMBR1L is a transmembrane protein expressed on the plasma and ER membranes. It functions as a negative feedback regulator of Wnt signaling. In the ER of lymphocytes, a second Wnt/β-catenin pathway destruction complex exists, consisting of LMBR1L, GP78, and UBAC2. GP78 is an ER membrane-anchored E3 ubiquitin ligase that prevents accumulation of misfolded proteins via ERAD. UBAC2, a central element in the GP78 complex, contains a functional poly-UB-binding domain at its C-teriminus (UBA). The LMBR1LGP78UBAC2 complex ubiquitinates and prevents maturation of FZD6 and Wnt co-receptor LRP6 within the ER of lymphocytes, and the complex may also regulate the ubiquitination and degradation of β-catenin. Absent this second destruction complex, FZD6 and LRP6 accumulate on the plasma membrane and enhanced Wnt signaling overwhelms the canonical destruction complex, causing β-catenin to flood the nucleus. Additionally, LMBR1L physically interacts with several components of the destruction complex including GSK-3β. These interactions help to stabilize GSK-3β, which is needed for tonic inactivation of β-catenin by phosphorylation to attenuate canonical Wnt signaling. In LMBR1L-deficient lymphocytes, reduced expression of destruction complex proteins is observed with increased amounts of the inactive (phosphorylated) form of GSK-3β, suggesting destabilization of the complex. As a result, high levels of unphosphorylated β-catenin accumulate, triggering apoptosis in response to lymphocyte activation.

FIG. 29. Amino acid sequence alignment of human (SEQ ID NO. 3) and mouse LMBR1L (SEQ ID NO. 4). Identical residues are highlighted in red and similar residues are highlighted in yellow. NCBI gene accession number for human LMBR1L is NP_060583.2, and mouse LMBR1L is NP_083374.1.

FIG. 30A: Serum dsDNA-specific IgG levels in 4 to 6 months old Lmbr1l^{+/+} (n=6), Lmbr1l^{-/-} (n=4), Lmbr1l^{+/+}; Bcl2-Tg (n=11), Lmbr1l^{-/-}; Bcl2-Tg (n=3), and 6 months old NZB/NZW F1 hybrid females (n=4).

FIG. 30B: Peripheral blood B cell counts in 4 to 5 months old Lmbr1l^{+/+} (n=6), Lmbr1l^{-/-} (n=5), Lmbr1l^{+/+}; Bcl2-Tg (n=6), and Lmbr1l^{-/-}; Bcl2-Tg (n=7) mice. Each symbol represents an individual mouse. Error bars indicate S.D. *P<0.05; **P<0.01; ***P<0.001.

### DETAILED DESCRIPTION

It is to be understood that both the foregoing general description and the following detailed description are exemplary and explanatory only and are not restrictive of the compositions and methods of the present disclosure.

Disclosed herein are compositions and methods related to inhibiting LMBR1L (limb region 1 like) in a subject with conditions in which the immune system is excessive or overactive, such as inflammatory diseases, autoimmune diseases, graft versus host disease, or an allograft rejection, as well as kits that can be used in such methods. One aspect of the present disclosure relates to the surprising discovery that a mutation in LMBR1L, or a knockout of LMBR1L, causes a phenotype characterized by immunodeficiency, including decreased frequencies of CD3⁺ T cells in the peripheral blood, increased CD4⁺ to CD8⁺ ratio, increased surface glycoproteins CD44 and CD62L, impaired B cell development, diminished T cell-dependent and T cell-independent humoral immune responses, decreased cytotoxic T lymphocyte (CTL) killing activity, and reduced frequencies of natural killer (NK) and NK T cells. As such, LMBR1L is essential for lymphopoiesis. An inhibitor of LMBR1L, such as an antibody, can be used to reduce or suppress an immune response in a subject in need thereof.

LMBR1L is a multi-spanning plasma membrane protein, previously of unknown function. Unexpectedly, as disclosed herein, in the absence of LMBR1L, all lymphocyte dependent immunity was strongly suppressed, and lymphoid cells were driven to apoptosis by stimuli that typically cause proliferation. Also, surprisingly, the experiments of this disclosure demonstrate that LMBR1L is an essential component of the Wnt signaling pathway in lymphocytes of all lineages. Signaling via the Wnt pathway was abnormal in LMBR1L knockout mice, as β-catenin activity was constitutively high and the destruction complex could not engage (i.e., FRIZZLED-6 becomes highly upregulated and ZNRF3 was down-regulated at the cell membrane). The data of the present disclosure shows that LMBR1L can interact with several components of the Wnt signaling pathway in lymphocytes, including GSK3β, β-catenin, ZNRF3, RNF43, and FRIZZLED-6. Furthermore, it was unexpectedly discovered that LMBR1L deficiency inhibits autoimmune response such as the production of autoantibodies (dsDNA-specific IgG), which is a specific and sensitive indication for systemic lupus erythematosus and other autoimmune diseases.

Therefore, LMBR1L inhibitors, such as antibodies and small molecule antagonists, can be used for reducing or suppressing an immune response in a subject with conditions in which the immune system is excessive or overactive, e.g., inflammatory diseases, autoimmune diseases, graft versus host disease, or an allograft rejection.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs. The following references provide one of skill with a general definition of many of the terms used in this disclosure: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

“Lmbr1l” and “LMBR1L”, also known as LIMR, are used interchangeably and refer to limb region 1 like, with “Lmbr1l” generally referring to the gene or mRNA, and “LMBR1L” the protein product unless otherwise noted. It should be understood that the terms include the complete gene, the cDNA sequence, the complete amino acid sequence, or any fragment or variant thereof. In some embodiments, the LMBR1L is human LMBR1L.

As used herein, the term “LMBR1L inhibitor” is intended to include therapeutic agents that inhibit, down-modulate, suppress or down-regulate LMBR1L activity. The term is intended to include chemical compounds, such as small molecule inhibitors or antagonists and biologic agents (e.g., antibodies), interfering RNA (shRNA, siRNA), gene editing/silencing tools (CRISPR/Cas9, TALENs) and the like.

An “anti-LMBR1L antibody” is an antibody that immuno-specifically binds to LMBR1L (e.g., its extracellular domain). The antibody may be an isolated antibody. Such binding to LMBR1L exhibits a K_{d} with a value of, e.g., no greater than 1 μM, no greater than 100 nM, or no greater than 50 nM. K_{d} can be measured by any methods known to one skilled in the art, such as a surface plasmon resonance assay or a cell binding assay. An anti-LMBR1L antibody may be a monoclonal antibody or an antigen-binding fragment thereof.

An “antibody” as used herein is a protein consisting of one or more polypeptides comprising binding domains that bind to a target epitope. The term antibody includes monoclonal antibodies comprising immunoglobulin heavy and light chain molecules, single heavy chain variable domain antibodies, and variants and derivatives thereof, including chimeric variants of monoclonal and single heavy chain variable domain antibodies. Binding domains are substantially encoded by immunoglobulin genes or fragments of immunoglobulin genes, wherein the protein immuno-specifically binds to an antigen. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. For most vertebrate organisms, including humans and murine species, the typical immunoglobulin structural unit comprises a tetramer that is composed of two identical pairs of polypeptide chains, each pair having one “light” (about 25 kD) and one “heavy” chain (about 50-70 kD). “V_{L}” and V_{H}” refer to the variable domains of these light and heavy chains respectively. “C_{L}” and C_{H}″ refer to the constant domains of the light and heavy chains. Loops of β-strands, three each on the V_{L} and V_{H}, are responsible for binding to the antigen and are referred to as the “complementarity determining regions” or “CDRs”. The “Fab” (fragment, antigen-binding) region includes one constant and one variable domain from each heavy and light chain of the antibody, i.e., V_{L}, C_{L}, V_{H}, and C_{H}1.

Antibodies include intact immunoglobulins as well as antigen-binding fragments thereof. The term “antigen-binding fragment” refers to a polypeptide fragment of an antibody which binds antigen or competes with intact antibody (i.e., with the intact antibody from which they were derived) for antigen binding (i.e., specific binding). Antigen binding fragments can be produced by recombinant or biochemical methods that are well known in the art. Exemplary antigen-binding fragments include Fv, Fab, Fab′, (Fab′)₂, CDR, paratope, and single chain Fv antibodies (scFv) in which a V_{H} and a V_{L} chain are joined together (directly or through a peptide linker) to form a continuous polypeptide.

Antibodies also include variants, chimeric antibodies, and humanized antibodies. The term “antibody variant” as used herein refers to an antibody with single or multiple mutations in the heavy chains and/or light chains. In some embodiments, the mutations exist in the variable region. In some embodiments, the mutations exist in the constant region. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example. “Humanized” antibodies refer to a molecule having an antigen-binding site that is substantially derived from an immunoglobulin from a non-human species and the remaining immunoglobulin structure of the molecule based upon the structure and/or sequence of a human immunoglobulin. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, or six) which are altered with respect to the original antibody, which are also termed one or more CDRs “derived from” one or more CDRs.

As described herein, the amino acid residues of an antibody can be numbered according to the general numbering of Kabat (Kabat, et al. (1991) Sequences of Proteins of Immunological Interest, 5th edition. Public Health Service, NIH, Bethesda, Md.).

The term “binding” as used herein in the context of binding between an antibody, such as a VHH, and an epitope of LMBR1L as a target, refers to the process of a non-covalent interaction between molecules. Preferably, said binding is specific. The specificity of an antibody can be determined based on affinity. A specific antibody can have a binding affinity or dissociation constant K_{d} for its epitope of less than 10⁻⁷ M, preferably less than 10⁻⁸ M.

The term “affinity” refers to the strength of a binding reaction between a binding domain of an antibody and an epitope. It is the sum of the attractive and repulsive forces operating between the binding domain and the epitope. The term affinity, as used herein, refers to the dissociation constant, K_{d}.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping, and hydrogendeuterium exchange.

The site on the antibody that binds the epitope is referred to as “paratope,” which typically includes amino acid residues that are in close proximity to the epitope once bound. See Sela-Culang et al., Front Immunol. 2013; 4: 302.

“Immunohistochemistry” or “IHC” refers to the process of detecting an antigen in cells of a tissue section allowing the binding and subsequent detection of antibodies immunospecifically recognizing the antigen of interest in a biological tissue. For a review of the IHC technique, see, e.g., Ramos-Vara et al., Veterinary Pathology January 2014 vol. 51 no. 1, 42-87, incorporated herein by reference in its entirety. To evaluate IHC results, different qualitative and semi-quantitative scoring systems have been developed. See, e.g., Fedchenko et al., Diagnostic Pathology, 2014; 9: 221, incorporated herein by reference in its entirety. One example is the H-score, determined by adding the results of multiplication of the percentage of cells with staining intensity ordinal value (scored from 0 for “no signal” to 3 for “strong signal”) with 300 possible values.

“Immunospecific” or “immunospecifically” (sometimes used interchangeably with “specifically”) refer to antibodies that bind via domains substantially encoded by immunoglobulin genes or fragments of immunoglobulin genes to one or more epitopes of a protein of interest, but which do not substantially recognize and bind other molecules in a sample containing a mixed population of antigenic molecules. Typically, an antibody binds immunospecifically to a cognate antigen with a K_{d} with a value of no greater than 50 nM, as measured by a surface plasmon resonance assay or a cell binding assay. The use of such assays is well known in the art.

The term “immune response” includes T cell mediated responses, B cell mediated immune responses, and/or NK cell mediated responses, as well as changes in the number and/or development of T, B and/or NK cells (e.g., by regulating common lymphoid progenitors). In addition, the term immune response includes immune responses that are indirectly affected by T cell activation, e.g., antibody production (humoral responses) and activation of cytokine responsive cells, e.g., macrophages.

As used herein, the term “lymphopoiesis” has its general meaning in the art and refers to the generation of lymphocytes such as B, T and NK cells. Thus, the term “T-cell lymphopoiesis” refers to the generation of T cells (i.e. T lymphocytes).

The term “autoimmune disease” refers to a disease that arises from an overactive immune response in a subject, in which the subject's immune system produces antibodies that attack the subject's own cells, leading to the deterioration, and in some cases, the destruction of cells and/or tissue. Examples of autoimmune diseases include, without limitation, Type 1 diabetes, Multiple Sclerosis, coeliac disease, lupus erythematosus, systemic lupus erythematosus (SLE), Sjogren's syndrome, Churg-Strauss Syndrome, Hashimoto's thyroiditis, Graves' disease, idiopathic thrombocytopenic purpura, rheumatoid arthritis (RA), ankylosing spondylitis, Crohn's disease, dermatomyositis, Goodpasture's syndrome, Guillain-Barre syndrome (GBS), mixed Connective tissue disease, myasthenia gravis, narcolepsy, pemphigus vulgaris, pernicious anaemia, psoriasis, psoriatic arthritis, polymyositis, primary biliary cirrhosis, relapsing polychondritis, temporal arteritis, ulcerative colitis, vasculitis, and Wegener's granulomatosis. The term “inflammatory disease” as used herein is defined as a disorder that results from an excessive inflammatory response (or inflammatory overresponse). An inflammatory disease is the result of an inappropriate and excessive response to an inappropriate antigen. Examples of inflammatory diseases include but are not limited to, allergy, asthma, autoimmune diseases, coeliac disease, glomerulonephritis, hepatitis, inflammatory bowel disease, rheumatoid arthritis, lupus, preperfusion injury, transplant rejection, Addison's disease, alopecia areata, dystrophic epidermolysis bullosa, epididymitis, vasculitis, vitiligo, myxedema, pernicious anemia, and ulcerative colitis, among others. Inflammatory Bowel Disease (IBD) includes two major types, namely Crohn's Disease (CD) and Ulcerative Colitis (UC).

The term “agent” can include any molecule, peptide, antibody or other agent which can reduce or suppress an immune response in a subject with conditions in which the immune system is overactive, such as an inflammatory disease, autoimmune disease, graft versus host disease, or an allograft rejection. Various agents are useful in the compositions and methods described herein.

The terms “cross-compete”, “cross-competition”, “cross-block”, “cross-blocked,” and “cross-blocking” are used interchangeably herein to mean the ability of an antibody or fragment thereof to interfere with the binding directly or indirectly through allosteric modulation of the anti-LMBR1L antibodies of the present disclosure to the target LMBR1L. The extent to which an antibody or fragment thereof is able to interfere with the binding of another to the target, and therefore whether it can be said to cross-block or cross-compete according to the present disclosure, can be determined using competition binding assays. One particularly suitable quantitative cross-competition assay uses a FACS- or an AlphaScreen-based approach to measure competition between the labelled (e.g. His tagged, biotinylated or radioactive labelled) antibody, or fragment thereof, and the other an antibody or fragment thereof in terms of their binding to the target. In general, a cross-competing antibody or fragment thereof is, for example, one which can bind to the target in the cross-competition assay such that, during the assay and in the presence of a second antibody or fragment thereof, the recorded displacement of the immunoglobulin single variable domain or polypeptide according to the disclosure is up to 100% (e.g., in FACS based competition assay) of the maximum theoretical displacement (e.g., displacement by cold (e.g., unlabeled) antibody or fragment thereof that needs to be cross-blocked) by the to be tested potentially cross-blocking antibody or fragment thereof that is present in a given amount. Preferably, cross-competing antibodies or fragments thereof have a recorded displacement that is between 10% and 100%, more preferred between 50% to 100%.

The terms “suppress”, “suppression”, “inhibit”, “inhibition”, “neutralize,” and “neutralizing” as used interchangeably herein, refer to any statistically significant decrease in biological activity (e.g., LMBR1L activity), including full blocking of the activity. For example, “inhibition” can refer to a decrease of about 10%, 20%, 30%, 40%, 50%, 60%, 70%, 80%, 90%, or 100% in biological activity.

The term “subject” or “patient” includes a human or other mammalian animal that receives either prophylactic or therapeutic treatment.

The terms “treat,” “treating,” and “treatment” as used herein refer to therapeutic or preventative measures such as those described herein. The methods of “treatment” employ administration to a patient a LMBR1L inhibitor provided herein, for example, a patient with conditions in which the immune system is overactive, such as an inflammatory disease, graft-versus-host disease, allograft rejection, or an autoimmune disease (e.g., Hashimoto's thyroiditis, Grave's Disease, type I insulin-dependent diabetes, rheumatoid arthritis (RA), systemic lupus erythematosus (SLE), multiple sclerosis (MS)), in order to prevent, cure, delay, reduce the severity of, or ameliorate one or more symptoms in the patient with conditions in which the immune system is excessive or overactive, or in order to prolong the survival of a patient beyond that expected in the absence of such treatment.

The term “effective amount,” as used herein, refers to that amount of an agent, such as a LMBR1L inhibitor, for example an anti-LMBR1L antibody, which is sufficient for reducing or suppressing an immune response in a patient with conditions in which the immune system is excessive or overactive, e.g., an inflammatory disease, autoimmune disease, graft versus host disease, or an allograft rejection, and/or effect treatment, prognosis, or diagnosis of an inflammatory disease, autoimmune disease, graft versus host disease, or an allograft rejection, when administered to a patient. A therapeutically effective amount will vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg of an antibody or antigen binding portion thereof, as provided herein. Dosing may be, e.g., every week, every 2 weeks, every three weeks, every 4 weeks, every 5 weeks or every 6 weeks. Dosage regimens may be adjusted to provide the optimum therapeutic response. An effective amount is also one in which any toxic or detrimental effects (side effects) of the agent are minimized and/or outweighed by the beneficial effects. Administration may be intravenous at exactly or about 6 mg/kg or 12 mg/kg weekly, or 12 mg/kg or 24 mg/kg biweekly. Additional dosing regimens are described below.

Other terms used in the fields of recombinant nucleic acid technology, microbiology, immunology, antibody engineering, and molecular and cell biology as used herein will be generally understood by one of ordinary skill in the applicable arts. For example, conventional techniques may be used for preparing recombinant DNA, performing oligonucleotide synthesis, and practicing tissue culture and transformation (e.g., electroporation, transfection or lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Various aspects and embodiments are described in further detail in the following subsections.

### LMBR1L

The limb region 1 like (LMBR1L) is a nine-transmembrane spanning cell surface protein, and as disclosed herein, is required for normal function of all lymphoid lineages, including T cells, B cells, NK and NK T cells. LMBR1L has been identified as a receptor for the small secretory protein, human Liopcalin-1 (PMID: 23964685). The full gene sequence of human LMBR1L is 14,985 bp in length (GenBank ID No. NC_000012.12). Prior to the present disclosure, the LMBR1L protein was genetically linked to multiple congenital limb malformations. However, further studies of the human and mouse loci showed that the original association with limb defects was incidental because of the disruption of a long-range SHH enhancer located within an intron of LMBR1L (Dolezal, D. Proc Natl Acad Sci USA. 2015 Nov. 10; 112(45): 13928-13933). Prior to the present disclosure, the function of LMBR1L had not been elucidated.

As described herein, a phenotype was detected in a forward genetic screen associated with cell-autonomous failure of all lymphoid lineages in mice. The causative mutation was identified in Lmbr1l, which encodes a nine-spanning membrane protein with no previously described function in immunity. LMBR1L deficiency in T cells increased expression of the Wnt co-receptor frizzled-6 (FZD6) and low-density lipoprotein receptor-related protein 6 (LRP6), resulting in aberrant activation of the Wnt/β-catenin pathway and apoptotic cell death upon stimulation. Interaction of LMBR1L with ubiquitin-associated domain containing 2 (UBAC2) and glycoprotein 78 (GP78) causes downregulation of Wnt signaling in lymphocytes by preventing maturation of FZD6 and LRP6 through ubiquitination within the endoplasmic reticulum. The present disclosure thus establishes an essential function for LMBR1L during lymphopoiesis and lymphoid activation, in which it acts as a negative regulator of the Wnt/β-catenin pathway.

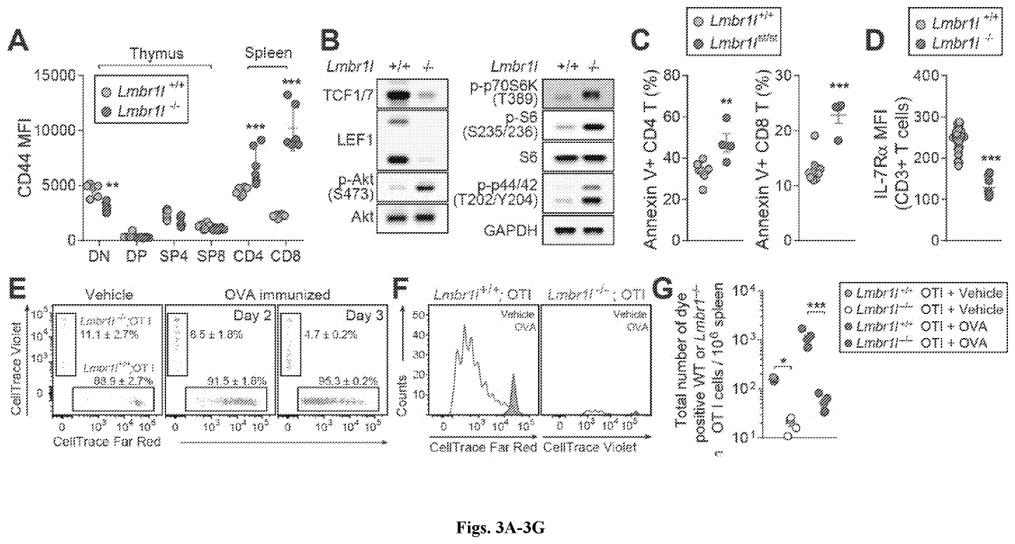

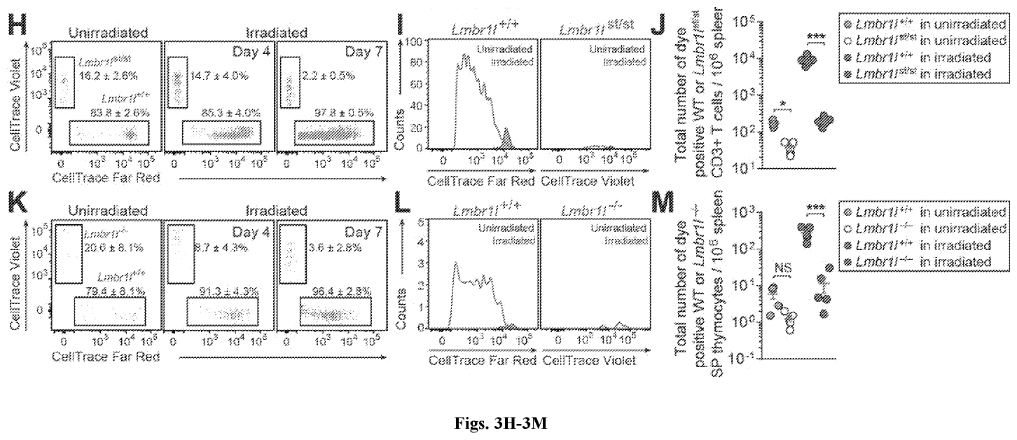

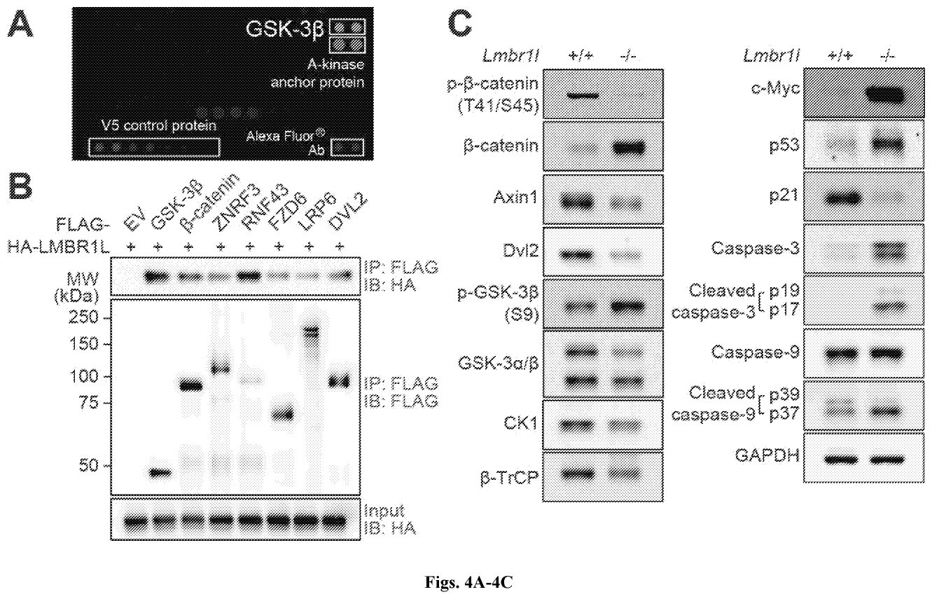

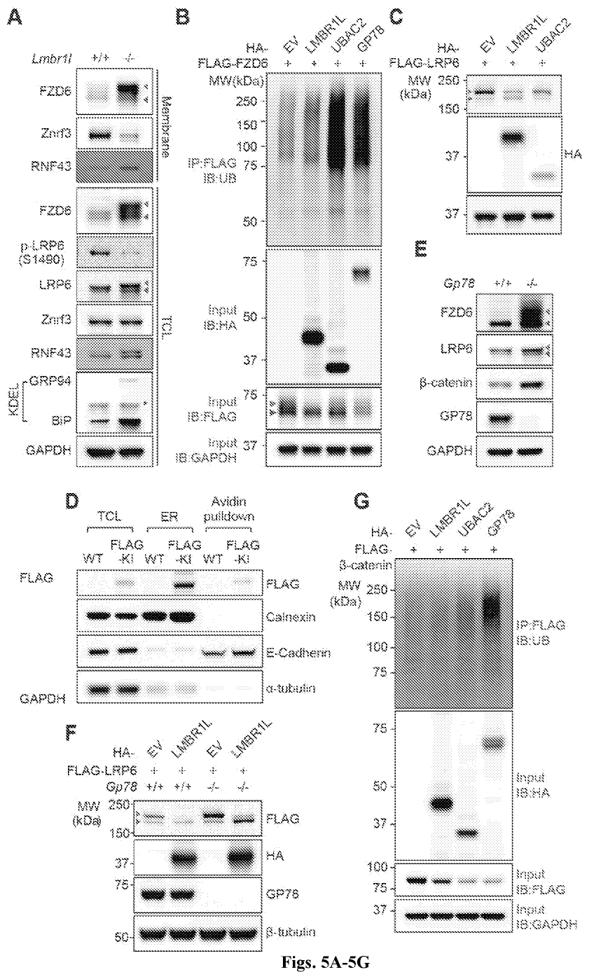

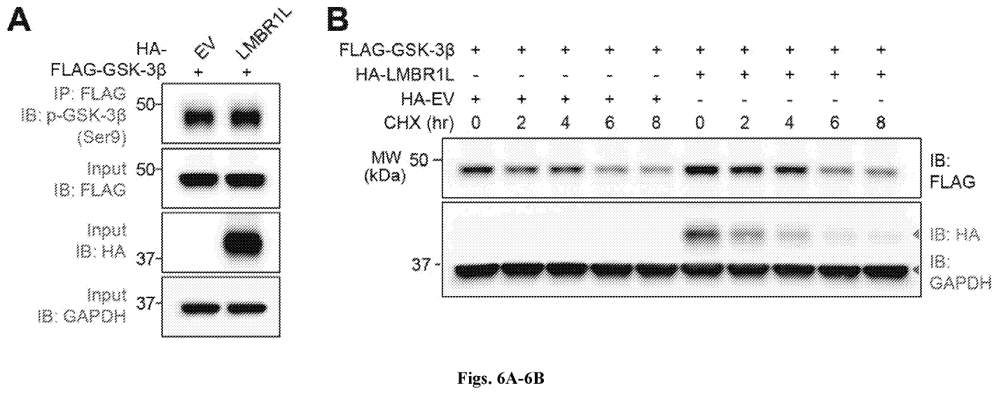

Also disclosed herein are LMBR1L interacting proteins. Four of the proteins are essential components of the endoplasmic reticulum-associated degradation (ERAD) pathway, including ubiquitin associated domain containing 2 (UBAC2), transitional endoplasmic reticulum ATPase (TERA known as VCP), UBX domain-containing protein 8 (UBXD8, known as FAF2), and Glycoprotein 78 (GP78; known as AMFR). Components of the Wnt/β-catenin signaling pathway are also identified herein as putative LMBR1L interactors, including zinc and ring finger 3 (ZNRF3), low-density lipoprotein receptor-related protein 6 (LRP6), β-catenin, glycogen synthase kinase-3a (GSK3a), and GSK3β. Additional analysis confirmed that LMBR1L interacts with each of the Wnt and ERAD components (FIGS. 3B and 13), suggesting that LMBR1L might be a critical component of the ERAD and Wnt/β-catenin signaling pathways. Indeed, LMBR1L has been identified herein as a novel negative regulator of Wnt/β-catenin signaling. LMBR1L exists in the GP78-UBAC2 complex to attenuate Wnt/β-catenin signaling by inhibiting Wnt co-receptor maturation within the ER.

The findings herein demonstrate the existence of a previously unrecognized pathway that regulates Wnt/β-catenin signaling in lymphocytes. The exaggerated apoptosis of T cells that results in lymphopenia stems from aberrant activation of Wnt/β-catenin signaling in LMBR1L-deficient mice. In the absence of LMBR1L, mature forms of Wnt co-receptors are highly upregulated and components of the destruction complex are downregulated. These alterations contribute to the accumulation of β-catenin, which enters the nucleus and promotes the transcription of target genes such as c-Myc, p53, and CD44. This signal transduction cascade favors apoptosis in an intrinsic and extrinsic caspase cascade-dependent manner.

As such, by inhibiting LMBR1L, immunosuppression can be achieved. This is particularly useful for treating a disease or condition where the subject's immune system is overactive. Compositions for inhibiting LMBR1L and thus, reducing or suppressing an immune response in a subject with conditions in which the immune system is excessive or overactive are also provided. The composition can include one or more anti-LMBR1L antibodies disclosed herein, or an antigen binding fragment thereof. In some embodiments, other LMBR1L inhibitors, such as small molecule compounds, can also be used to inhibit one or more activities of LMBR1L.

Furthermore, LMBR1L deficiency has been shown as a possible etiology in previously unexplained pan-lymphoid immunodeficiency disorders. Thus, compositions and methods for treating an immunodeficiency disorder can include introducing a nucleic acid (DNA or mRNA) such as a transgene encoding LMBR1L into a subject in need thereof.

### LMBR1L Inhibitors and Uses Thereof

Inhibition of LMBR1L can reduce or suppress an immune response in a subject with conditions in which the immune system is excessive or overactive, such as an inflammatory disease, graft-versus-host disease, allograft rejection, or an autoimmune disease (e.g., Hashimoto's thyroiditis, Grave's Disease, type I insulin-dependent diabetes, rheumatoid arthritis (RA), systemic lupus erythematosus (SLE), multiple sclerosis (MS)). As such, LMBR1L inhibitors can be used as an effective agent in an immunosuppressive therapy. Without wishing to be bound by theory, it is believed that LMBR1L deficiency or inhibition can lead to apoptosis of lymphocytes such as T cells, as well as inhibition of autoimmune responses such as the production of autoantibodies (e.g., dsDNA-specific IgG). LMBR1L has an essential function during lymphopoiesis and lymphoid activation, acting as a negative regulator of the Wnt/β-catenin pathway.

Various LMBR1L inhibitors are included in the present disclosure. Examples include chemical compounds, such as small molecule inhibitors and biologic agents (e.g., antibodies) that can bind LMBR1L and inhibit or decrease its activity, e.g., measured in a Western Blot Analysis or ZNRF3, FRIZZLED-6, β-catenin, and/or c-Myc expression assay. Agents that regulate Lmbr1l gene expression level are also included, such as interfering RNA (shRNA, siRNA) and gene editing/silencing tools (CRISPR/Cas9, TALENs, zinc finger nucleases) that are designed specifically to target the Lmbr1l gene or a regulatory sequence thereto.

In some embodiments, a method for identifying an LMBR1L inhibitor is provided, which can include contacting a cell with a test agent, wherein an increase in expression of FRIZZLED-6, β-catenin, and/or c-Myc, and/or a decrease in expression of ZNRF3, compared to a control cell that is not contacted with the test agent indicates that the test agent is an LMBR1L inhibitor.

In some embodiments, the LMBR1L inhibitor can be characterized by at least partial inhibition of proliferation (e.g., by at least 10% relative to control) of a cell expressing LMBR1L.

In certain embodiments, the LMBR1L inhibitor is an anti-LMBR1L antibody, e.g., a monoclonal antibody, or an antigen-binding fragment thereof. In certain embodiments, the anti-LMBR1L antibody can be a modified antibody, e.g., chimeric or humanized antibody derived from a mouse anti-LMBR1L antibody. Methods for making modified antibodies are known in the art. In some embodiments, the anti-LMBR1L antibody is an antibody or antigen binding fragment thereof which binds to an epitope present on the human LMBR1L protein, e.g., the extracellular ectodomain, or a portion thereof.

In yet another embodiment, the LMBR1L inhibitor such as anti-LMBR1L antibody can comprise a mixture, or cocktail, of two or more anti-LMBR1L antibodies, each of which binds to a different epitope on LMBR1L. In one embodiment, the mixture, or cocktail, comprises three anti-LMBR1L antibodies, each of which binds to a different epitope on LMBR1L.

In another embodiment, the LMBR1L inhibitor can include a nucleic acid molecule, such as an RNA molecule, that inhibits the expression or activity of LMBR1L. Interfering RNAs specific for LMBR1L, such as shRNAs or siRNAs that specifically inhibit the expression and/or activity of LMBR1L, can be designed in accordance with methods known in the art.

In one aspect, use of an LMBR1L inhibitor for the manufacture of a medicament to reduce or suppress an immune response in a subject with conditions in which the immune system is excessive or overactive, such as an inflammatory disease, autoimmune disease, graft versus host disease, or an allograft rejection, is provided. In another aspect, a method of suppressing an immune response in a patient with conditions in which the immune system is excessive or overactive, such as an inflammatory disease, autoimmune disease, graft versus host disease, or an allograft rejection, is provided, the method comprising administering to the patient an effective amount of an LMBR1L inhibitor.

### Preparation of Anti-LMBR1L Antibodies

Anti-LMBR1L antibodies can be made using various methods generally known in the art. For example, phage display technology can be used to screen a human antibody library to produce a fully human monoclonal antibody for therapy. High affinity binders can be considered candidates for neutralization studies. Alternatively, a conventional monoclonal approach can be used, in which mice or rabbits can be immunized with the human protein, candidate binders identified and tested, and a humanized antibody ultimately produced by engrafting the combining sites of heavy and light chains into a human antibody encoding sequence.

Antibodies typically comprise two identical pairs of polypeptide chains, each pair having one full-length “light” chain (typically having a molecular weight of about 25 kDa) and one full-length “heavy” chain (typically having a molecular weight of about 50-70 kDa). The amino-terminal portion of each chain typically includes a variable region of about 100 to 110 or more amino acids that typically is responsible for antigen recognition. The carboxy-terminal portion of each chain typically defines a constant region responsible for effector function. The variable regions of each of the heavy chains and light chains typically exhibit the same general structure comprising four relatively conserved framework regions (FR) joined by three hyper variable regions, also called complementarity determining regions or CDRs. The CDRs from the two chains of each pair typically are aligned by the framework regions, which alignment may enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (1987 and 1991, National Institutes of Health, Bethesda, Md.), Chothia & Lesk, 1987, J Mol. Biol. 196:901-917, or Chothia et al., 1989, Nature 342:878-883).

Antibodies became useful and of interest as pharmaceutical agents with the development of monoclonal antibodies. Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, Marcel Dekker, Inc., New York, pp. 51-63).

Monoclonal antibodies may be modified for use as therapeutics. One example is a “chimeric” antibody in which a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Other examples are fragments of such antibodies, so long as they exhibit the desired biological activity. See, U.S. Pat. No. 4,816,567; and Morrison et al. (1985), Proc. Natl. Acad. Sci. USA 81:6851-6855. A related development is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass.

Another development is the “humanized” antibody. Methods for humanizing non-human antibodies are well known in the art (see U.S. Pat. Nos. 5,585,089, and 5,693,762; see also Cecile Vincke et al. J. Biol. Chem. 2009; 284:3273-3284 for humanization of llama antibodies). Generally, a humanized antibody is produced by a non-human animal, and then certain amino acid residues, typically from non-antigen recognizing portions of the antibody, are modified to be homologous to said residues in a human antibody of corresponding isotype. Humanization can be performed, for example, using methods described in the art (Jones et al., 1986, Nature 321:522-525; Riechmann et al., 1988, Nature 332:323-327; Verhoeyen et al., 1988, Science 239:1534-1536), by substituting at least a portion of a rodent variable region for the corresponding regions of a human antibody.

More recent is the development of human antibodies without exposure of antigen to human beings (“fully human antibodies”). Using transgenic animals (e.g., mice) that are capable of producing a repertoire of human antibodies in the absence of endogenous mouse immunoglobulin production, such antibodies are produced by immunization with an antigen (typically having at least 6 contiguous amino acids), optionally conjugated to a carrier. See, for example, Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of these methods, transgenic animals are produced by incapacitating the endogenous mouse immunoglobulin loci encoding the mouse heavy and light immunoglobulin chains therein, and inserting loci encoding human heavy and light chain proteins into the genome thereof. Partially modified animals, which have less than the full complement of modifications, are then cross-bred to obtain an animal having all of the desired immune system modifications. When administered an immunogen, these transgenic animals produce antibodies that are immunospecific for these antigens having human (rather than murine) amino acid sequences, including variable regions. See PCT Publication Nos. WO96/33735 and WO94/02602, incorporated by reference. Additional methods are described in U.S. Pat. No. 5,545,807, PCT Publication Nos. WO91/10741, WO90/04036, and in EP 546073B1 and EP 546073A1, incorporated by reference. Human antibodies may also be produced by the expression of recombinant DNA in host cells or by expression in hybridoma cells as described herein.

In some embodiments, phage display technology may be used to screen for therapeutic antibodies. In phage display, antibody repertoires can be displayed on the surface of filamentous bacteriophage, and the constructed library may be screened for phages that bind to the immunogen. Antibody phage is based on genetic engineering of bacteriophages and repeated rounds of antigen-guided selection and phage propagation. This technique allows in vitro selection of LMBR1L monoclonal antibodies. The phage display process begins with antibody-library preparation followed by ligation of the variable heavy (VH) and variable light (VL) PCR products into a phage display vector, culminating in analysis of clones of monoclonal antibodies. The VH and VL PCR products, representing the antibody repertoire, are ligated into a phage display vector (e.g., the phagemid pComb3X) that is engineered to express the VH and VL as an scFv fused to the pIII minor capsid protein of a filamentous bacteriophage of Escherichia coli that was originally derived from the M13 bacteriophage. However, the phage display vector pComb3X does not have all the other genes necessary to encode a full bacteriophage in E. coli. For those genes, a helper phage is added to the E. coli that are transformed with the phage display vector library. The result is a library of phages, each expressing on its surface a LMBR1L monoclonal antibody and harboring the vector with the respective nucleotide sequence within. The phage display can also be used to produce the LMBR1L monoclonal antibody itself (not attached to phage capsid proteins) in certain strains of E. Coli. Additional cDNA is engineered, in the phage display vector, after the VL and VH sequences to allow characterization and purification of the mAb produced. Specifically, the recombinant antibody may have a hemagglutinin (HA) epitope tag and a polyhistidine to allow easy purification from solution.

Diverse antibody phage libraries are produced from ˜10⁸ independent E. coli transformants infected with helper phage. Using bio-panning, a library can be screened for phage binding to the immunogen sequence listed above, or a fragment thereof, through the expressed surface of the monoclonal antibody. Cyclic panning allows for pulling out potentially very rare antigen-binding clones and consists of multiple rounds of phage binding to antigen (immobilized on ELISA plates or in solution on cell surfaces), washing, elution, and reamplification of the phage binders in E. coli. During each round, specific binders are selected out from the pool by washing away non-binders and selectively eluting binding phage clones. After three or four rounds, highly specific binding of phage clones through their surface LMBR1L monoclonal antibody is characteristic for directed selection on the immobilized immunogen.

Another method is to add a C-terminal His tag, suitable for purification by affinity chromatography, to the immunogen sequence listed above. Purified protein can be inoculated into mice together with a suitable adjuvant. Monoclonal antibodies produced in hybridomas can be tested for binding to the immunogen, and positive binders can be screened as described in the assays herein.

Fully human antibodies can also be produced from phage-display libraries (as disclosed in Hoogenboom et al., 1991, J. Mol. Biol. 227:381; and Marks et al., 1991, J. Mol. Biol. 222:581). These processes mimic immune selection through the display of antibody repertoires on the surface of filamentous bacteriophage and subsequent selection of phage by their binding to an antigen of choice. One such technique is described in PCT Publication No. WO99/10494, incorporated by reference, which describes the isolation of high affinity and functional agonistic antibodies for MPL- and msk-receptors using such an approach.

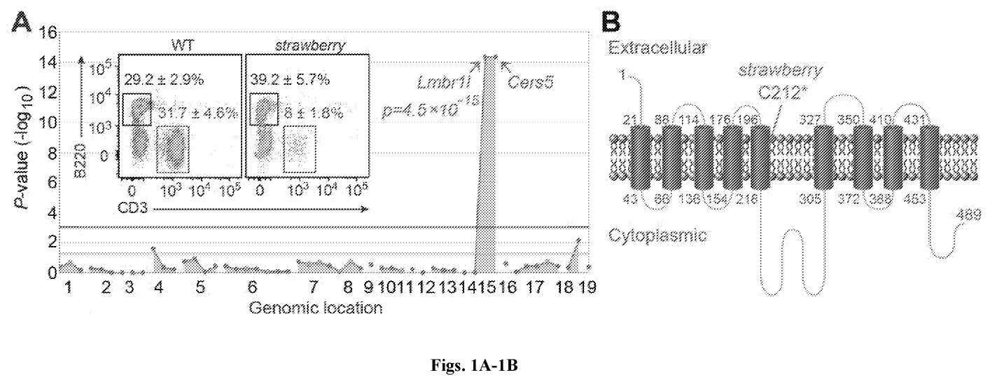

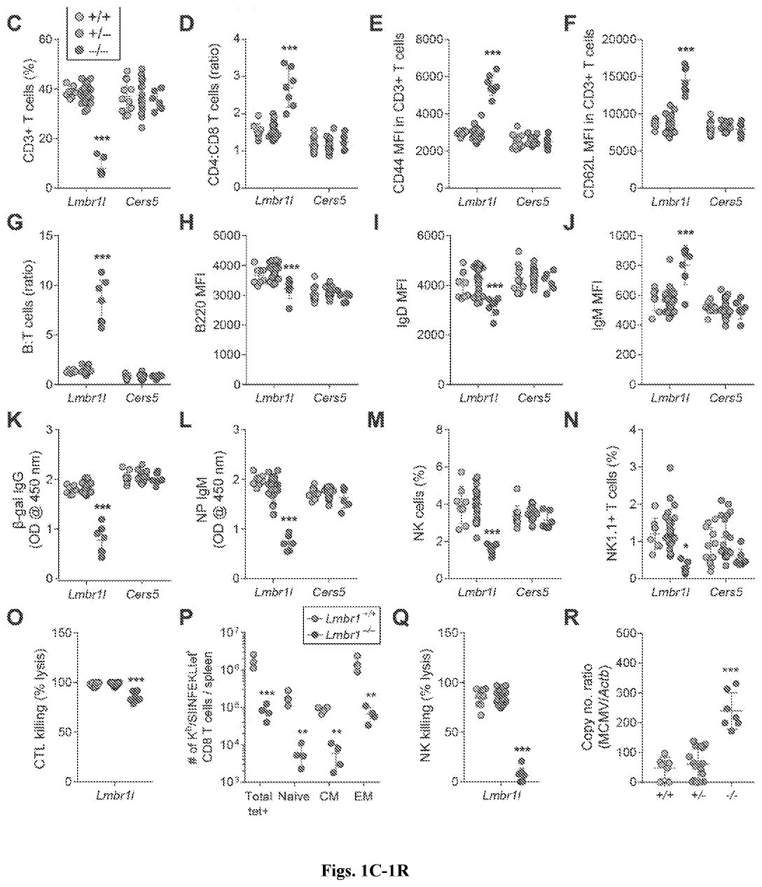

In some embodiments, the extracellular domains of human LMBR1L can be used as the immunogen. The human LMBR1L has five extracellular domains (FIG. 1B). These extracellular domains include:

(1) MEAPDYEVLSVREQLFHERIR (SEQ ID NO. 5);

(2) SNEVLLSLPRNYYIQWLNGSLIHGLWN (SEQ ID NO. 6);

(3) VDKNKANRESLYDFWEYYLPY (SEQ ID NO. 7);

(4) DEAAMPRGMQGTSLGQVSFSKLGS (SEQ ID NO. 8); and

(5) SRTLGLTRFDLLGDFGRFNWLG (SEQ ID NO. 9).

A fragment or portion of the human LMBR1L extracellular domain can also be used as the immunogen. Monoclonal antibodies can be raised using one or more immunogens. Potential therapeutic anti-LMBR1L antibodies can be generated.

In one example, using a mouse model having one or more extracellular domains of human LMBR1L knocked into the mouse Lmbr1l gene, and human T cells (Jurkat or primary T cells from human donors), monoclonal antibodies that phenocopy the knockout mutation can be tested and identified as potential anti-LMBR1L antibody candidates. Monoclonal antibodies that phenocopy the knockout mutation can display phenotypes such as a reduced number of T cells (e.g., CD4+ and CD8+), B cells, NK and/or NK T cells. Such tests include screening endpoint(s), such as the augmentation of FRIZZLED-6, ZNRF3, β-catenin and/or c-Myc protein expression detected on, e.g., Western blot. After the screening, fully human monoclonal antibodies can be developed for preclinical testing and then tested in clinical human trials for safety and efficacy. Such antibodies can be clinical candidates that can reduce or suppress an immune response in a subject with conditions in which the immune system is excessive or overactive, such as an inflammatory disease, autoimmune disease, graft versus host disease, or an allograft rejection, and/or improve immune suppressive therapy (e.g., by decreasing the number of lymphocytes).

Nucleotide sequences encoding the above antibodies can be determined. Thereafter, chimeric, CDR-grafted, humanized, and fully human antibodies also may be produced by recombinant methods. Nucleic acids encoding the antibodies can be introduced into host cells and expressed using materials and procedures generally known in the art.

The disclosure provides one or more monoclonal antibodies against LMBR1L. Preferably, the antibodies bind to one or more extracellular domains, or fragments thereof, of human LMBR1L. In preferred embodiments, the disclosure provides nucleotide sequences encoding, and amino acid sequences comprising, heavy and light chain immunoglobulin molecules, particularly sequences corresponding to the variable regions thereof. In preferred embodiments, sequences corresponding to CDRs, specifically from CDR1 through CDR3, are provided. In additional embodiments, the disclosure provides hybridoma cell lines expressing such immunoglobulin molecules and monoclonal antibodies produced therefrom, preferably purified human monoclonal antibodies against human LMBR1L.

The CDRs of the light and heavy chain variable regions of anti-LMBR1L antibodies of the disclosure can be grafted to framework regions (FRs) from the same, or another, species. In certain embodiments, the CDRs of the light and heavy chain variable regions of anti-LMBR1L antibody may be grafted to consensus human FRs. To create consensus human FRs, FRs from several human heavy chain or light chain amino acid sequences are aligned to identify a consensus amino acid sequence. The FRs of the anti-LMBR1L antibody heavy chain or light chain can be replaced with the FRs from a different heavy chain or light chain. Rare amino acids in the FRs of the heavy and light chains of anti-LMBR1L antibody typically are not replaced, while the rest of the FR amino acids can be replaced. Rare amino acids are specific amino acids that are in positions in which they are not usually found in FRs. The grafted variable regions from anti-LMBR1L antibodies of the disclosure can be used with a constant region that is different from the constant region of anti-LMBR1L antibody. Alternatively, the grafted variable regions are part of a single chain FAT antibody. CDR grafting is described, e.g., in U.S. Pat. Nos. 6,180,370, 5,693,762, 5,693,761, 5,585,089, and 5,530,101, which are hereby incorporated by reference for any purpose.

In some embodiments, antibodies of the disclosure can be produced by hybridoma lines. In these embodiments, the antibodies of the disclosure bind to LMBR1L with a dissociation constant (K_{d}) of between approximately 4 pM and 1 μM. In certain embodiments of the disclosure, the antibodies bind to LMBR1L with a K_{d} of less than about 100 nM, less than about 50 nM or less than about 10 nM.

In preferred embodiments, the antibodies of the disclosure are of the IgG1, IgG2, or IgG4 isotype, with the IgG1 isotype most preferred. In preferred embodiments of the disclosure, the antibodies comprise a human kappa light chain and a human IgG1, IgG2, or IgG4 heavy chain. In particular embodiments, the variable regions of the antibodies are ligated to a constant region other than the constant region for the IgG1, IgG2, or IgG4 isotype. In certain embodiments, the antibodies of the disclosure have been cloned for expression in mammalian cells.

In alternative embodiments, antibodies of the disclosure can be expressed in cell lines other than hybridoma cell lines. In these embodiments, sequences encoding particular antibodies can be used for transformation of a suitable mammalian host cell. According to these embodiments, transformation can be achieved using any known method for introducing polynucleotides into a host cell, including, for example, packaging the polynucleotide in a virus (or into a viral vector) and transducing a host cell with the virus (or vector) or by transfection procedures known in the art. Such procedures are exemplified by U.S. Pat. Nos. 4,399,216, 4,912,040, 4,740,461, and 4,959,455 (all of which are hereby incorporated herein by reference for any purpose). Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene-mediated transfection, protoplast fusion, electroporation, encapsulation of the polynucleotide(s) in liposomes, and direct microinjection of the DNA into nuclei.

According to certain embodiments of the methods of the disclosure, a nucleic acid molecule encoding the amino acid sequence of a heavy chain constant region, a heavy chain variable region, a light chain constant region, or a light chain variable region of a LMBR1L antibody of the disclosure is inserted into an appropriate expression vector using standard ligation techniques. In a preferred embodiment, the LMBR1L heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y.

Typically, expression vectors used in any of the host cells can contain sequences for plasmid maintenance and for cloning and expression of exogenous nucleotide sequences. Such sequences typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These sequences are well known in the art.

Expression vectors of the disclosure may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an anti-LMBR1L antibody has been inserted into the proper site of the vector, the completed vector may be inserted into a suitable host cell for amplification and/or polypeptide expression. The transformation of an expression vector for an anti-LMBR1L antibody into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., supra.

The host cell, when cultured under appropriate conditions, synthesizes an anti-LMBR1L antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, one may select cell lines by determining which cell lines have high expression levels and produce antibodies with constitutive LMBR1L binding properties. In another embodiment, one may select a cell line from the B cell lineage that does not make its own antibody but has a capacity to make and secrete a heterologous antibody (e.g., mouse myeloma cell lines NS0 and SP2/0).

### Pharmaceutical Compositions and Use Thereof

In another aspect, pharmaceutical compositions are provided that can be used in the methods disclosed herein, i.e., pharmaceutical compositions for reducing or suppressing an immune response in a subject with conditions in which the immune system is excessive or overactive, such as an inflammatory disease, autoimmune disease, graft versus host disease, or an allograft rejection, and/or improve immune suppressive therapy (e.g., by decreasing the number of lymphocytes).

In some embodiments, the pharmaceutical composition comprises an LMBR1L inhibitor and a pharmaceutically acceptable carrier. The LMBR1L inhibitor can be formulated with the pharmaceutically acceptable carrier into a pharmaceutical composition. Additionally, the pharmaceutical composition can include, for example, instructions for use of the composition for the treatment of patients to reduce or suppress an immune response in a subject with conditions in which the immune system is overactive, and/or improve immune suppressive therapy.

In one embodiment, the LMBR1L inhibitor can be an anti-LMBR1L antibody or antigen-binding fragment thereof.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions comprising an LMBR1L inhibitor can be administered alone or in combination therapy. For example, the combination therapy can include a composition provided herein comprising an LMBR1L inhibitor and at least one or more additional therapeutic agents, such as one or more chemotherapeutic agents known in the art, discussed in further detail below. Pharmaceutical compositions can also be administered in conjunction with radiation therapy and/or surgery.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg, and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks), and once a month.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (e.g., 0.005 to 70%, e.g., 0.01 to 30%) of active ingredient in combination with a pharmaceutically acceptable carrier.

In certain embodiments, the methods and uses provided herein for reducing or suppressing an immune response in a subject with conditions in which the immune system is excessive or overactive, and/or improve immune suppressive therapy (e.g., by decreasing the number of lymphocytes), can comprise administration of an LMBR1L inhibitor and at least one additional agent that is not an LMBR1L inhibitor.

In one aspect, the improved effectiveness of a combination according to the disclosure can be demonstrated by achieving therapeutic synergy.

The term “therapeutic synergy” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on parameter tumor volume.

The term “additive” refers to when the combination of two or more products at given doses is equally efficacious than the sum of the efficacies obtained with of each of the two or more products, whilst the term “superadditive” refers to when the combination is more efficacious than the sum of the efficacies obtained with of each of the two or more products. Disclosed herein are compositions and methods for reducing or suppressing an immune response in a subject with conditions in which the immune system is overactive. The method includes inhibiting LMBR1L in a subject in need thereof. In certain embodiments, inhibiting LMBR1L can reduce the number of T cells (e.g., CD4+ and CD8+), B cells, NK and/or NK T cells, thereby providing an immune suppressive therapy. LMBR1L inhibition (e.g., an anti-LMBR1L antibody) can be used as a stand-alone immune suppressive therapy by e.g., reducing the number of lymphocytes in a subject. In some embodiments, LMBR1L inhibition can be used in conjunction with other therapies.

In various embodiments, the methods disclosed herein can include administering to the subject an effective amount of LMBR1L inhibitor such as anti-LMBR1L antibody or antigen-binding fragment thereof. In general, the effective amount can be administered therapeutically and/or prophylactically.

Treatment can be suitably administered to subjects, particularly humans, suffering from, having, susceptible to, or at risk of developing such cancer. Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g. opinion) or objective (e.g. measurable by a test or diagnostic method).

### Administration of the Formulation

The formulations of the present disclosure, including, but not limited to, reconstituted and liquid formulations, are administered to a mammal in need of treatment with the LMBR1L inhibitors disclosed herein, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

In preferred embodiments, the formulations are administered to the mammal by subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to LMBR1L inhibitors, the format of the formulation used, and the discretion of the attending physician. The LMBR1L inhibitor is suitably administered to the patient at one time or over a series of treatments and may be administered to the patient at any time from diagnosis onwards. The LMBR1L inhibitor may be administered as the sole treatment or in conjunction with other drugs or therapies useful in treating the condition in question.

For LMBR1L inhibitors, an initial candidate dosage can range from about 0.1-20 mg/kg for administration to the patient, which can take the form of one or more separate administrations. However, other dosage regimens may be useful. The progress of such therapy is easily monitored by conventional techniques.

According to certain embodiments of the present disclosure, multiple doses of an LMBR1L inhibitor (or a pharmaceutical composition comprising a combination of LMBR1L inhibitor and any of the additional therapeutically active agents mentioned herein) may be administered to a subject over a defined time course. The methods according to this aspect of the disclosure comprise sequentially administering to a subject multiple doses of an LMBR1L inhibitor such as anti-LMBR1L antibody of the disclosure. As used herein, “sequentially administering” means that each dose of LMBR1L inhibitor is administered to the subject at a different point in time, e.g., on different days separated by a predetermined interval (e.g., hours, days, weeks or months). The present disclosure includes methods which comprise sequentially administering to the patient a single initial dose of an LMBR1L inhibitor, followed by one or more secondary doses of the LMBR1L inhibitor, and optionally followed by one or more tertiary doses of the LMBR1L inhibitor. The LMBR1L inhibitor may be administered at a dose of between 0.1 mg/kg to about 100 mg/kg.

The terms “initial dose,” “secondary doses,” and “tertiary doses” refer to the temporal sequence of administration of the LMBR1L inhibitor of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of LMBR1L inhibitor, but generally may differ from one another in terms of frequency of administration. In certain embodiments, however, the amount of LMBR1L inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of LMBR1L inhibitor which is administered to a patient prior to the administration of the very next dose in the sequence with no intervening doses.

The methods according to this aspect of the disclosure may comprise administering to a patient any number of secondary and/or tertiary doses of an LMBR1L inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

### Therapeutic Uses and Methods

The compositions disclosed herein (e.g., LMBR1L inhibitors) have numerous therapeutic utilities, including, e.g., the treatment of conditions or diseases where the immune system displays an excessive or overactive response. The present disclosure provides, inter alia, methods for reducing or suppressing an immune response in a subject with conditions in which the immune system is excessive or overactive, such as an inflammatory disease, autoimmune disease, graft versus host disease, or an allograft rejection. Exemplary methods comprise administering to the subject a therapeutically effective amount of any of the LMBR1L inhibitors described herein to provide, e.g., an immunosuppressive therapy.

Exemplary applications of immunosuppressive therapy include allo-immune diseases, auto-immune diseases, allergy, and other inflammatory diseases. Allo-immune diseases include organ transplant rejection, graft versus host disease (GVHD) (e.g., post allogeneic hematopoietic stem cell transplant, HSCT) and GVHD post allogeneic stem cell transplantation (SCT).

Autoimmune diseases are diseases in which the immune system attacks its own proteins, cells, and tissues. A comprehensive listing and review of autoimmune diseases can be found in The Autoimmune Diseases (Rose and Mackay, 2014, Academic Press). Exemplary autoimmune diseases that can be treated include Type 1 diabetes, Multiple Sclerosis, coeliac disease, lupus erythematosus, systemic lupus erythematosus (SLE), Sjogren's syndrome, Churg-Strauss Syndrome, Hashimoto's thyroiditis, Graves' disease, idiopathic thrombocytopenic purpura, rheumatoid arthritis (RA), ankylosing spondylitis, Crohn's disease, dermatomyositis, Goodpasture's syndrome, Guillain-Barre syndrome (GBS), mixed Connective tissue disease, myasthenia gravis, narcolepsy, pemphigus vulgaris, pernicious anaemia, psoriasis, psoriatic arthritis, polymyositis, primary biliary cirrhosis, relapsing polychondritis, temporal arteritis, ulcerative colitis, vasculitis, and Wegener's granulomatosis.

Inflammatory diseases can be used to broadly define a vast array of disorders and conditions that are characterized by inflammation. Examples include allergy, asthma, autoimmune diseases, coeliac disease, glomerulonephritis, hepatitis, inflammatory bowel disease, rheumatoid arthritis, lupus, preperfusion injury, transplant rejection, Addison's disease, alopecia areata, dystrophic epidermolysis bullosa, epididymitis, vasculitis, vitiligo, myxedema, pernicious anemia, and ulcerative colitis, among others. Inflammatory Bowel Disease (IBD) includes two major types, namely Crohn's Disease (CD) and Ulcerative Colitis (UC).

### EXAMPLES

The following examples, including the experiments conducted and results achieved, are provided for illustrative purposes only and are not to be construed as limiting the disclosure.

### Example 1: LMBR1L Regulates Lymphopoiesis Through Wnt/β-Catenin Signaling

Abstract: Precise control of Wnt signaling is necessary for immune system development. Here we detected severely impaired development of all lymphoid lineages in mice resulting from a N-ethyl-N-nitrosourea-induced mutation in limb region 1 like (Lmbr1l), encoding a membrane-spanning protein with no previously described function in immunity. Interaction of LMBR1L with glycoprotein 78 (GP78) and ubiquitin-associated domain containing 2 (UBAC2) attenuated Wnt signaling in lymphocytes by preventing the maturation of FZD6 and LRP6 through ubiquitination within the endoplasmic reticulum and by stabilizing destruction complex proteins. LMBR1L-deficient T cells exhibited hallmarks of Wnt/β-catenin activation and underwent apoptotic cell death in response to proliferative stimuli. LMBR1L has an essential function during lymphopoiesis and lymphoid activation, acting as a negative regulator of the Wnt/β-catenin pathway.

Introduction: The hematopoietic system consists of many cell types with specialized functions. Blood cells, derived from either the lymphoid or the myeloid lineage, are generated from hematopoietic stem cells (HSCs). HSCs continuously replenish all blood cell classes through a series of lineage-restricted steps and balance these mechanisms to maintain steady-state hematopoiesis throughout the lifetime of the organism. In the last two decades, canonical Wnt signaling (also known as Wnt/β-catenin signaling) and non-canonical Wnt signaling (e.g. the planar cell polarity pathway and Wnt-Ca′ signaling) have emerged as important regulators of the immune system by regulating HSC self-renewal, T and B cell development, and T cell activation (1-4). In lymphocytes, Wnt proteins function as growth-promoting factors but also affect cell-fate decisions including apoptosis and quiescence (5). Aberrant activation of the Wnt/β-catenin pathway in T cell lineages by deletion of adenomatous polyposis coli (Apc) causes T cell lymphopenia as a result of spontaneous activation and apoptosis of mature T cells in the periphery (6).

Given its widespread importance, several feedback regulatory mechanisms help to control proper Wnt signaling. These include the negative feedback regulator zinc and ring finger 3 (ZNRF3) and its homologue ring finger 43 (RNF43) (7). These transmembrane E3 ligases specifically promote the ubiquitination of lysine residues in the cytoplasmic loops of frizzled proteins (FZD), subjecting FZD to lysosomal degradation and thereby attenuating Wnt signaling (8, 9). Recently, disheveled (DVL) has been suggested as a critical intermediary for ZNRF3/RNF43-mediated ubiquitination and degradation of FZD (10). Loss of ZNRF3 and RNF43 expression is predicted to result in hyper-responsiveness to Wnt stimulation, and mutations in ZNRF3 and RNF43 have been observed in a variety of cancers in humans (7, 9). Despite the importance of Wnt signaling in immunity, negative feedback regulators that specifically control lymphopoiesis remain unknown. Here we describe the function and mechanism of action of LMBR1L in the negative regulation of Wnt signaling in lymphocytes.

### Immunodeficiency Caused by a Lmbr1l Mutation

To discover non-redundant regulators of lymphopoiesis and immunity, we carried out a forward genetic screen in mice carrying N-ethyl-N-nitrosourea (ENU)-induced mutations. We identified several mice descended from a common ENU-treated founder with low percentages of CD3⁺ T cells in the peripheral blood (inset in FIG. 1A). The phenotype, which we called strawberry (st), was transmitted as a recessive trait. By single-pedigree mapping, a method that analyzes genotype versus phenotype associations from a pedigree (11), the strawberry phenotype correlated with mutations in Lmbr1l and Cers5 (FIG. 1A). Lmbr1l encodes limb region 1 like (LMBR1L), a transmembrane protein of unknown function in immunity, and Cers5 encodes ceramide synthase 5 (CERS5), an enzyme in ceramide synthesis. The initial ambiguity concerning the causative effect of a mutation in Lmbr1l versus Cers5 was genetically resolved in favor of Lmbr1l (FIGS. 8A-8C). The Lmbr1l mutation in strawberry mice results in the substitution of cysteine 212 with a premature stop (C212*) in the fifth transmembrane helix of LMBR1L (FIG. 1B). This mutation was considered a putative null allele. CRISPR/Cas9-targeted knockout mutations of both Cers5 and Lmbr1l were generated, confirming that the mutation in Lmbr1l was solely responsible for the observed phenotype (FIG. 1C).

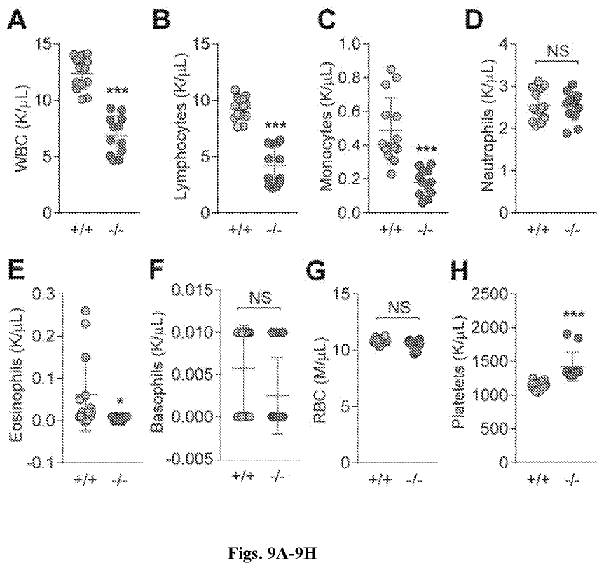

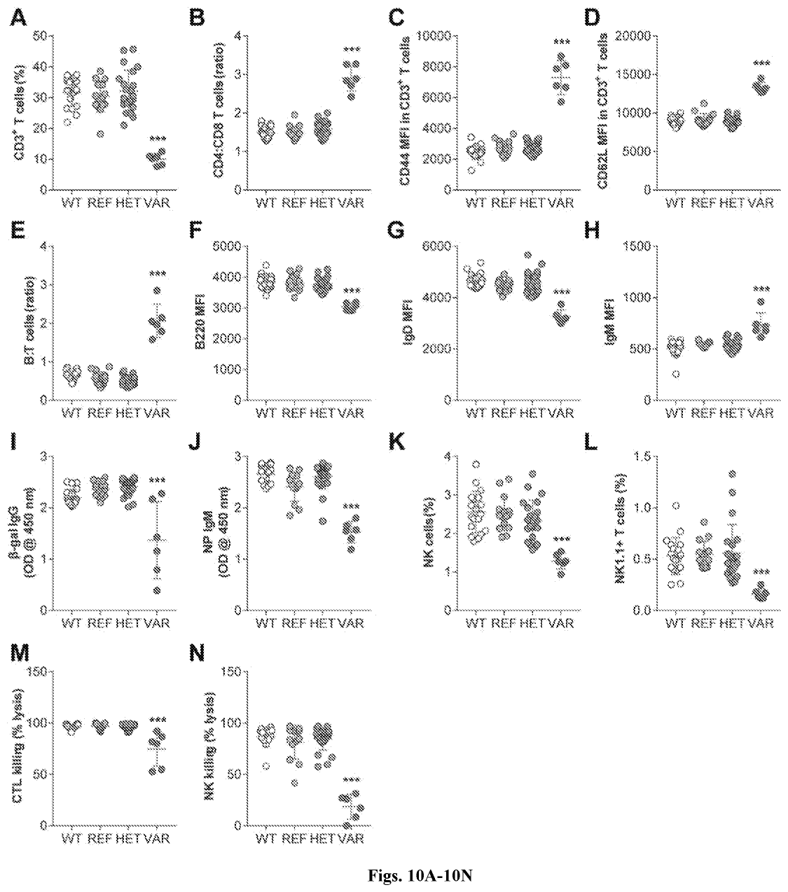

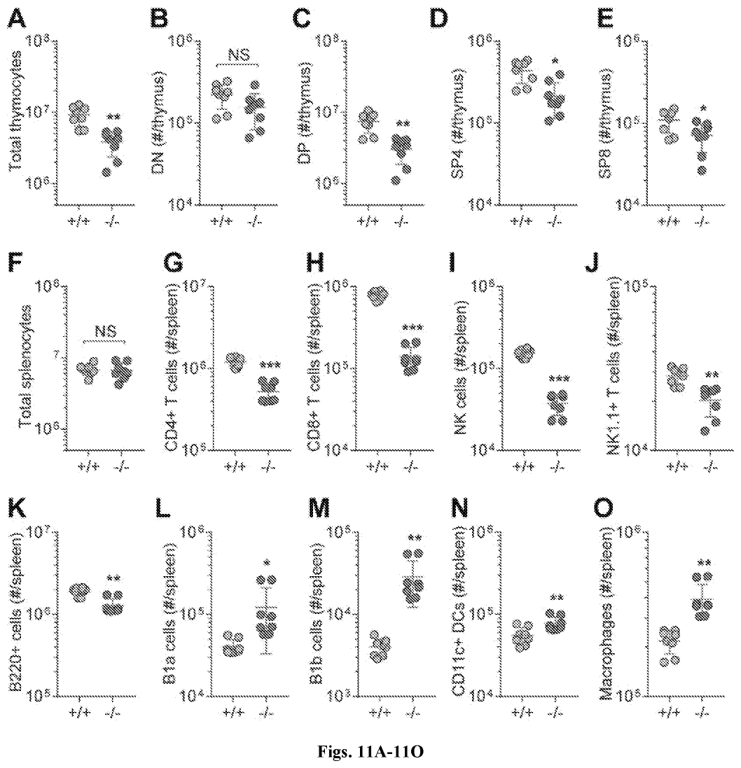

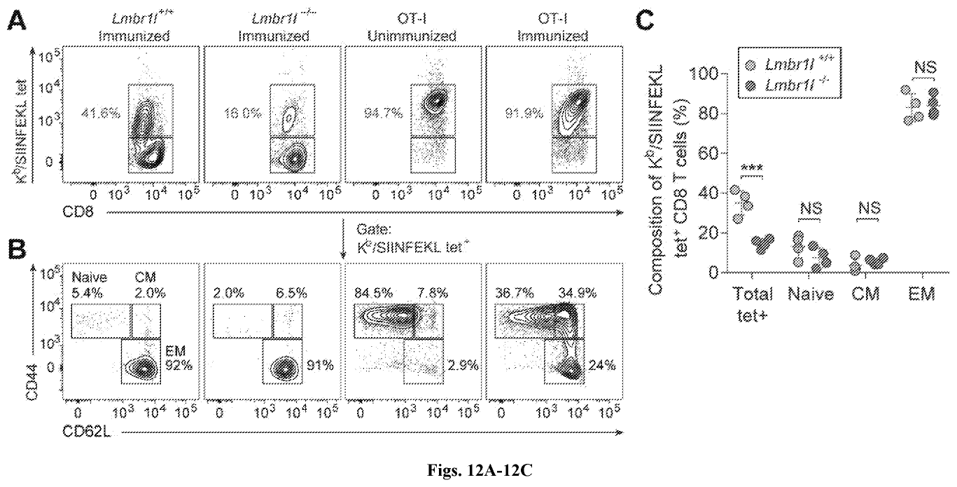

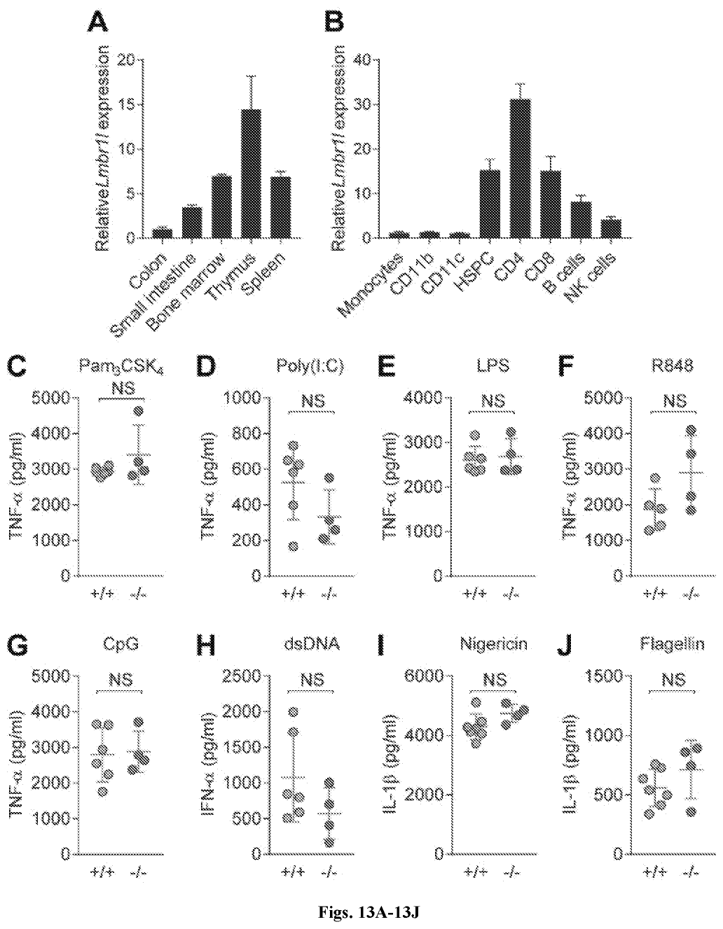

To further characterize the immunological defect caused by the Lmbr1l mutation, we immunophenotyped mice by complete blood count (CBC) testing, flow cytometric analysis of blood cells, immunization and analysis of antibody responses and memory formation, in vivo NK- and CTL-mediated cytotoxicity testing, and mouse cytomegalovirus infection (FIGS. 1C-1R, 9A-12C). The Lmbr1l^{-/-} mice were cytopenic with reduced numbers of leukocytes, lymphocytes, and monocytes (FIGS. 9A-9H). Consistent with the peripheral blood cell counts, Lmbr1l^{-/-} and strawberry mice had decreased frequencies of CD3⁺ T cells in the peripheral blood relative to those of wild-type littermates (FIGS. 1C, 10A). The CD4⁺-to-CD8⁺ T cell ratio was increased in Lmbr1l^{-/-} and strawberry mice (FIGS. 1D, 10B). The expression of surface glycoproteins CD44 and CD62L, which are abundant on expanding T cell populations, was increased (FIGS. 1E, 1F, 10C, 10D). The B cell-to-T cell ratio was also increased (FIGS. 1G, 10E). There was a reduction in surface B220 (FIGS. 1H, 10F) and IgD (FIGS. 1I, 10G) expression with a concomitant increase in IgM expression (FIGS. 1J, 10H) in the peripheral blood of Lmbr1l^{-/-} or strawberry homozygotes compared to wild-type mice. This suggests that the Lmbr1l mutation affected B cell development. The Lmbr1l^{-/-} mice had slightly smaller thymi compared to wild-type mice (FIG. 11A). The number of double-negative (DN) thymocytes were comparable between Lmbr1l^{-/-} and wild-type mice (FIG. 11B). However, we observed a decrease in double-positive (DP) and single-positive (SP) thymocytes in the Lmbr1l^{-/-} mice (FIGS. 11C-11E). Despite comparable numbers of total splenocytes, a marked reduction in the number of all lymphocytes was observed in Lmbr1l^{-/-} spleens compared to wild-type spleens (FIGS. 11F-11K). T cell-dependent and -independent humoral immune responses to recombinant Semliki Forest virus-encoded β-galactosidase (rSFV-(3 gal) and 4-hydroxy-3-nitrophenylacetyl-Ficoll (NP-Ficoll), respectively, were diminished (FIGS. 1K, 1L, 10I, 10J). The antigen-specific cytotoxic T lymphocyte (CTL) killing activity in immunized Lmbr1l^{-/-} or strawberry mice was also decreased compared to wild-type littermates (FIGS. 10, 10M). The antigen-specific CD8⁺ T cell response to immunization with aluminum hydroxide precipitated ovalbumin (OVA) was weaker in Lmbr1l^{-/-} mice compared to wild-type mice, as indicated by reduced total numbers (FIG. 1P) and frequencies of K^{b}/SIINFEKL-tetramer-positive CD8⁺ T cells (FIGS. 12A-12C) in the spleens of immunized Lmbr1l^{-/-} mice. The frequencies and numbers of natural killer (NK; FIGS. 1M, 10K, 11I) and NK1.1⁺ T cells (FIGS. 1N, 10L, 11J) were reduced in Lmbr1l^{-/-} or strawberry mice with a concomitant decrease in NK cell target killing (FIGS. 1Q, 10N). Furthermore, the Lmbr1l^{-/-} mice displayed susceptibility to mouse cytomegalovirus (MCMV) as determined by elevated viral titers in the liver (FIG. 1R) after challenge with a sublethal dose of MCMV. Lmbr1l mRNA was detected in a variety of mouse tissues and immune cells, with higher expression in the bone marrow, thymus, spleen, and lymphocytes (FIGS. 13A and 13B). However, LMBR1L deficiency had no effect on myeloid cell development (FIGS. 11N and 11O) or their function as determined by IFN-α, IL-1β, and TNF-α secretion in response to various stimuli (FIG. 13C-13J). Thus, LMBR1L is essential for lymphopoiesis.

### Cell-Intrinsic Failure of Lymphopoiesis

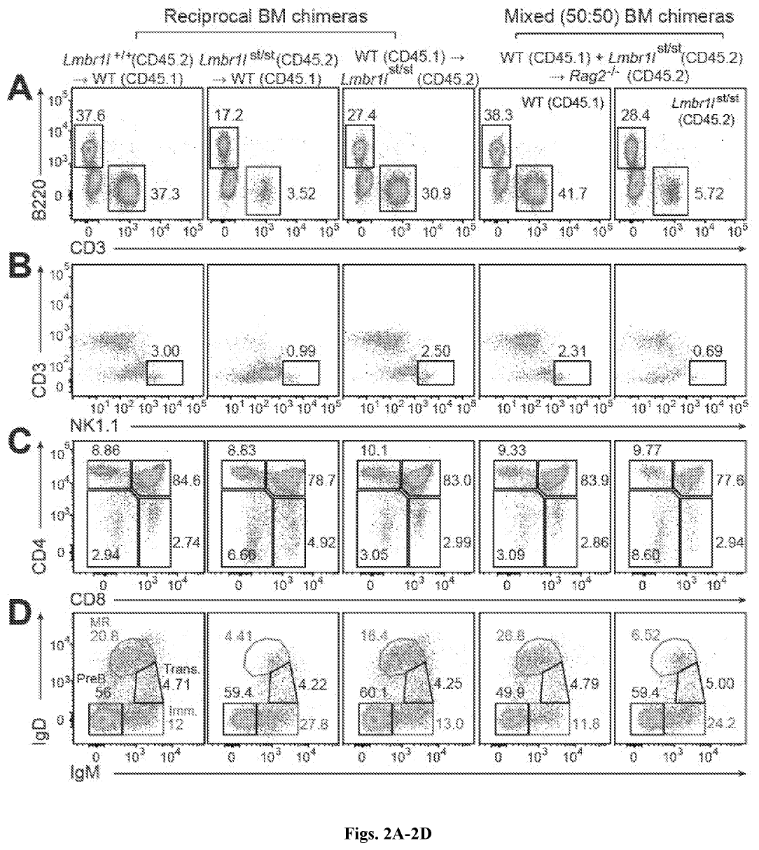

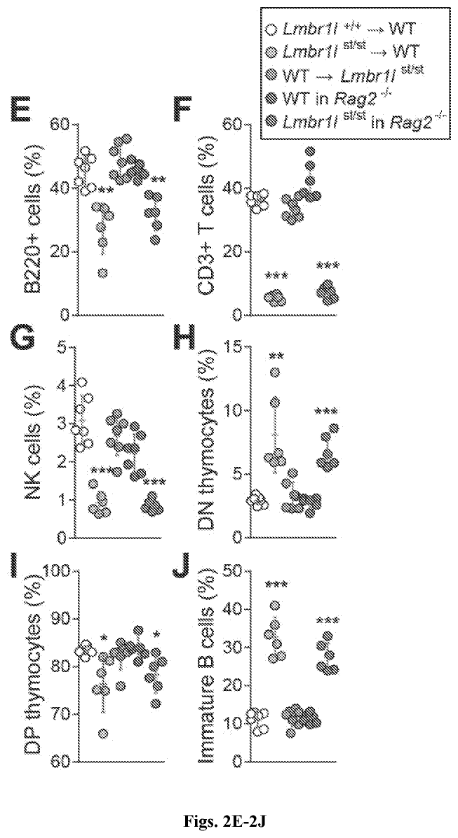

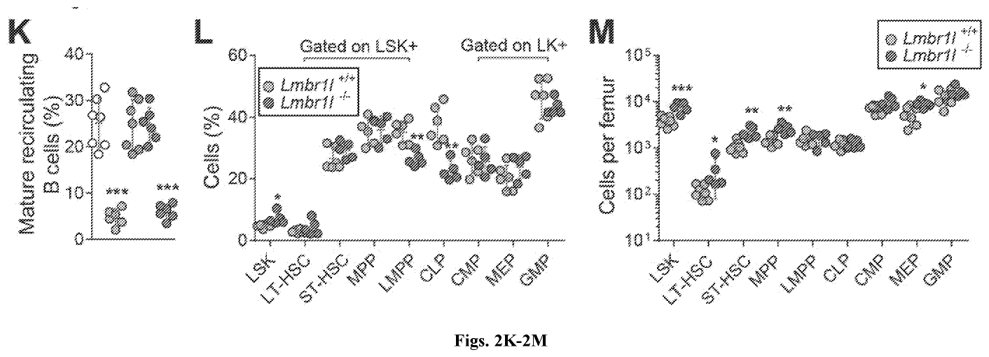

To determine the cellular origin of the Lmbr1l-associated defects, we reconstituted irradiated wild-type (CD45.1) or Rag2^{-/-} (CD45.2) recipients with unmixed wild-type (Lmbr1l^{+/+}; CD45.2) bone marrow, Lmbr1l mutant (CD45.2) bone marrow, or an equal mixture of mutant (CD45.2) and wild-type (CD45.1) bone marrow cells. In the absence or presence of competition, bone marrow cells from strawberry donors were unable to repopulate cells of lymphoid lineage such as B220⁺ (FIGS. 2A, 2E), CD3⁺ T (FIGS. 2A, 2F) and NK cells (FIGS. 2B, 2G) in the spleens of irradiated recipients as efficiently as cells derived from wild-type donors. The frequency of DN cells was increased and the frequency of DP cells was decreased in the thymus of mice that received strawberry bone marrow compared to those that received bone marrow from wild-type mice (FIGS. 2C, 2H, 2I), suggesting that the Lmbr1l mutation mildly affects T cell differentiation in the thymus.

In the bone marrow, immature B cells were increased among repopulated B cells derived from strawberry donors compared to those from wild-type donors (B220⁺IgM⁺IgD⁻; FIGS. 2D, 2J), and very few of the B cells from strawberry donors progressed to the mature recirculating B cell stage (B220⁺IgM⁺IgD⁺; FIGS. 2D, 2K). This developmental arrest occurred in both irradiated wild-type and Rag2^{-/-} recipients regardless of competition. We also detected decreased expression of B220 and IgD, and increased expression of IgM on peripheral blood B cells from strawberry homozygotes and Lmbr1l^{-/-} mice (FIGS. 1H-1J, 10F-10H). Thus, Lmbr1l mutations also impair B cell development.

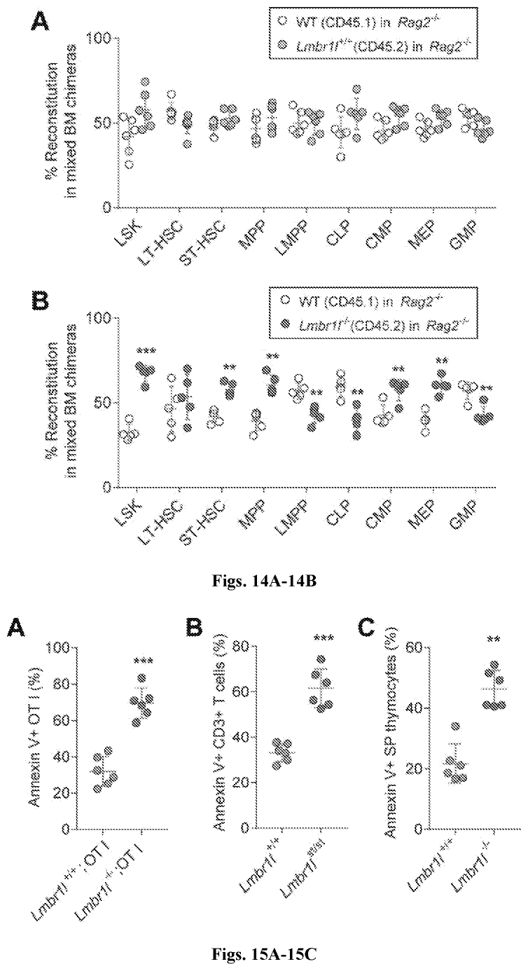

Lymphocytes, including B, T, and NK cells originate from lymphoid-primed multipotent progenitors (LMPPs) and common lymphoid progenitors (CLPs), which are thought to develop from LMPPs. Therefore, we examined the hematopoietic stem and progenitor cell populations in the bone marrow. LMBR1L deficiency caused an increase in the proportion and numbers of LSK⁺ cells compared to wild-type littermates (FIGS. 2L, 2M). The composition of the LSK compartment was mildly altered in Lmbr1l^{-/-} bone marrow, resulting in a reduction in the proportion of LMPPs and CLPs (FIG. 2L). In contrast, the numbers of long-term-hematopoietic stem cells (LT-HSCs), short-term (ST)-HSCs, and multipotent progenitors (MPPs) were increased in Lmbr1l^{-/-} bone marrow compared to those from wild-type mice (FIG. 2M). LMBR1L deficiency did not appreciably affect the composition and numbers of LK⁺ cells, including common myeloid progenitors (CMPs), megakaryocyteerythrocyte progenitors (MEPs), or granulocytemacrophage progenitors (GMPs; FIGS. 2L, 2M). Additionally, competitive bone marrow chimeras were made using a 1:1 mixture of Lmbr1l^{-/-} (CD45.2) and wild-type (CD45.1) bone marrow to assess the relative fitness of these progenitor populations. At 8 weeks post-transplant, Lmbr1l^{-/-}-derived hematopoietic cells were at an advantage in repopulating LSKs, ST-HSCs, MPPs, CMPs, and MEPs, while showing a disadvantage in repopulating LMPPs, CLPs, and GMPs (FIGS. 14A-14B). The observed HSC phenotype in Lmbr1l^{-/-} mice corresponds to the HSC phenotype when Wnt signaling is modestly increased in mice carrying hypomorphic Apc mutations (12). This suggests a specific effect of LMBR1L deficiency on lymphoid lineage commitment that is cell-autonomous.

Although the Lmbr1l mutation resulted in abnormal cellularity in the thymus as indicated by the increased proportion of DN cells together with decreased DP cells (FIGS. 2H, 2I), the remaining DP cells survived thymic selection and could develop into mature SP cells (FIG. 2C). Similar to peripheral blood T cells, CD4⁺ and CD8⁺ T cells in the spleens of Lmbr1l^{-/-} mice showed increased expression of the surface glycoprotein CD44, which encompasses recently activated, expanding, and memory phenotype cells (FIG. 3A). Increased CD44 expression was not evident in developing thymocytes (FIG. 3A). Immunoblot analysis of CD8⁺ T cells from Lmbr1l^{-/-} mice revealed T cell factor-1 (TCF-1) and lymphoid enhancer-binding factor 1 (LEF-1) downregulation, a phenotype previously observed in activated effector T cells (FIG. 3B) (13). Moreover, Akt, mitogen-activated protein kinase (p44/42 MAPK), p70S6K (a mTORC1 substrate), and ribosomal protein S6 (a p70S6K substrate), which are activated through phosphorylation, were constitutively phosphorylated under basal conditions in the CD8⁺ T cells from Lmbr1l^{-/-} mice (FIG. 3B). A higher percentage of CD4⁺ and CD8⁺ T cells from strawberry homozygotes were positive for annexin V under steady-state conditions compared to wild-type littermates (FIG. 3C). Lower IL-7Rα expression was seen in peripheral T cells of Lmbr1l^{-/-} mice compared to wild-type littermates (FIG. 3D). Thus, peripheral T cells from Lmbr1l mutant mice appear to exist in an activated state that may be predisposed to apoptosis, which led us to investigate their proliferative response to expansion signals.

To examine antigen-specific T cell proliferation, an equal mixture of OVA-specific wild-type (CD45.2) and Lmbr1l^{-/-} OT-I T cells (CD45.2) were transferred into wild-type recipients (CD45.1) that were subsequently immunized with soluble OVA. Wild-type OT-I T cells underwent proliferation as expected, but significantly fewer Lmbr1l^{-/-} OT-I T cells were detected in the spleen 2 or 3 days after immunization (FIGS. 3E-3G). We found that an excess of the Lmbr1l^{-/-} OT-I T cells were apoptotic, as indicated by annexin V staining (FIG. 15A). To further test the effect of the Lmbr1l mutation on T cell proliferation, we examined the response to homeostatic proliferation signals. An equal mixture of wild-type and homozygous strawberry splenic T cells was adoptively transferred into sublethally irradiated wild-type mice. Whereas wild-type T cells underwent extensive proliferation, homozygous strawberry T cells failed to proliferate in irradiated recipients (FIGS. 3H-3J) and showed a higher frequency of annexin V staining compared to wild-type T cells (FIG. 15B).

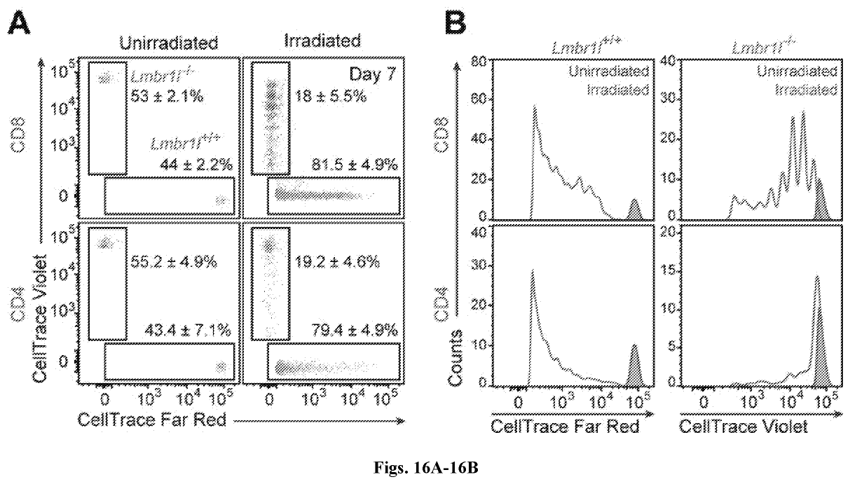

To address whether T cell homing to secondary lymphoid organs is impaired a mixture of wild-type and Lmbr1l^{-/-} dye-labeled pan T cells were transferred into irradiated recipients. A significant number of wild-type and Lmbr1l^{-/-} T cells were detected in the spleen of irradiated recipients after adoptive transfer, excluding the possibility of homing defects and further supporting that the Lmbr1l^{-/-} CD4⁺ and CD8⁺ T cells have proliferation defects (FIGS. 16A, 16B). These results demonstrate that Lmbr1l mutant or Lmbr1l^{-/-} T cells undergo apoptosis in response to antigen-specific or homeostatic expansion signals. To investigate whether the activated state (CD44^{hi}) of Lmbr1l^{-/-} T cells predisposes them to apoptosis, we isolated mature SP thymocytes (CD44^{lo}; FIG. 3A) and stimulated them to proliferate in response to homeostatic expansion signals. Similar to splenic T cells, mature SP thymocytes from Lmbr1l^{-/-} mice also failed to proliferate and showed an increased percentage of apoptotic cells (FIGS. 3K-3M, and 15C). Thus, LMBR1L-deficient T cells, regardless of activation state, die in response to expansion signals.

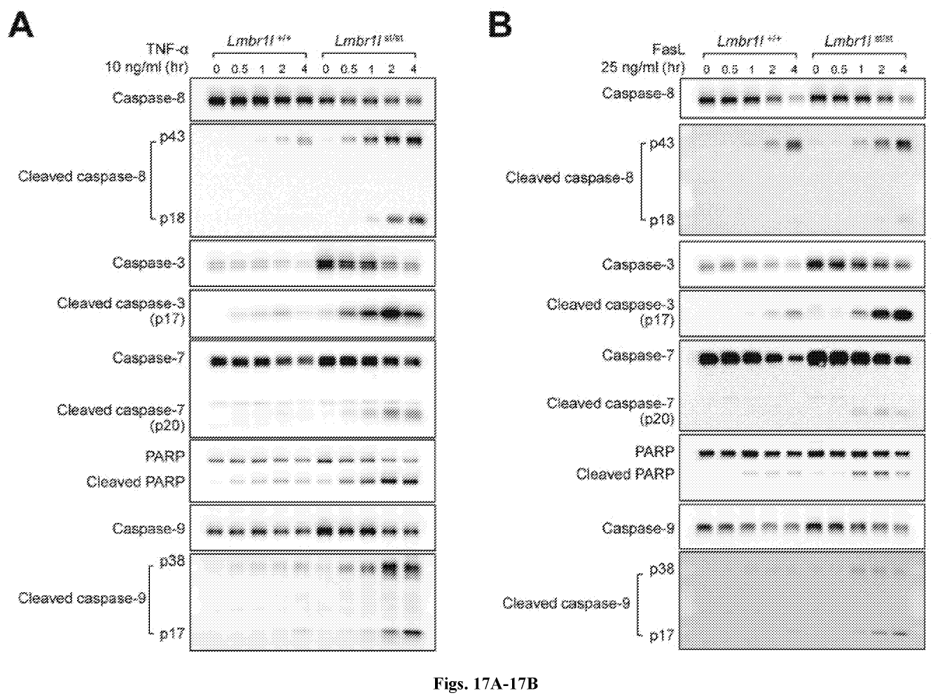

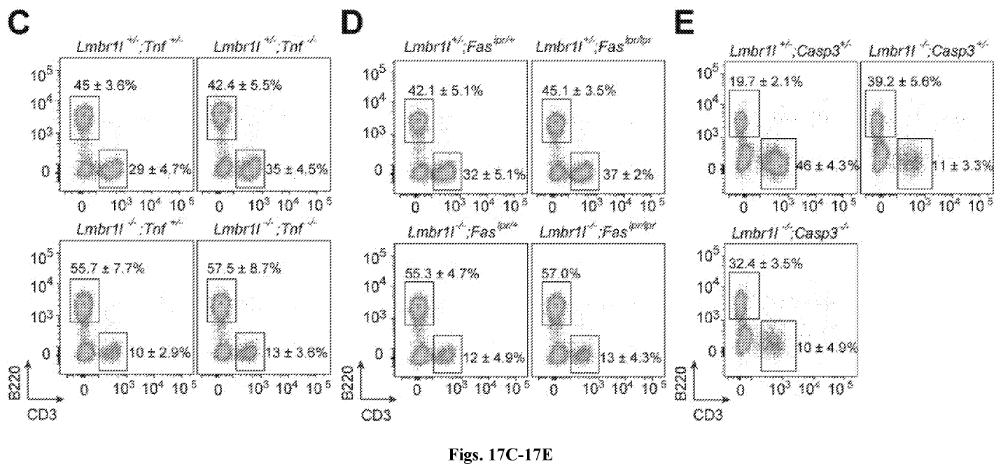

In the periphery, the balance between the expansion of activated (effector) T cells and their subsequent elimination during the termination of an immune response is controlled by extrinsic death receptors and caspase-dependent apoptosis, intrinsic mitochondria- and caspase-dependent apoptosis, or caspase-independent cell death. Treatment of either wild-type or Lmbr1l mutant CD8⁺ T cells with ligands for extrinsic death receptors such as tumor necrosis factor (TNF)-α or Fas ligand (FasL) enhanced proteolytic processing of caspases of the extrinsic apoptotic pathway (e.g., caspase-8, -3, -7 and PARP). Levels of cleaved caspases were increased in Lmbr1l mutant T cells relative to wild-type T cells (FIGS. 17A, 17B). Moreover, excessive cleavage of caspase-9, a key player in the intrinsic pathway, was detected in Lmbr1l^{-/-} cells after treatment with extrinsic apoptosis inducers (FIGS. 17A, 17B). Thus, both extrinsic and intrinsic caspase cascades appear to play roles in LMBR1L-deficient T cell apoptosis. Notably, deficiency of TNF-α (FIG. 17C), Fas (FIG. 17D), or caspase-3 (FIG. 17E) failed to rescue the T cell deficiency in Lmbr1l^{-/-} mice. Neither Fas-, TNFR-, nor caspase-3-mediated apoptosis pathways were solely responsible for the death of Lmbr1l^{-/-} T cells.

### Identification of LMBR1L as a Negative Regulator of Wnt/β-Catenin Signaling

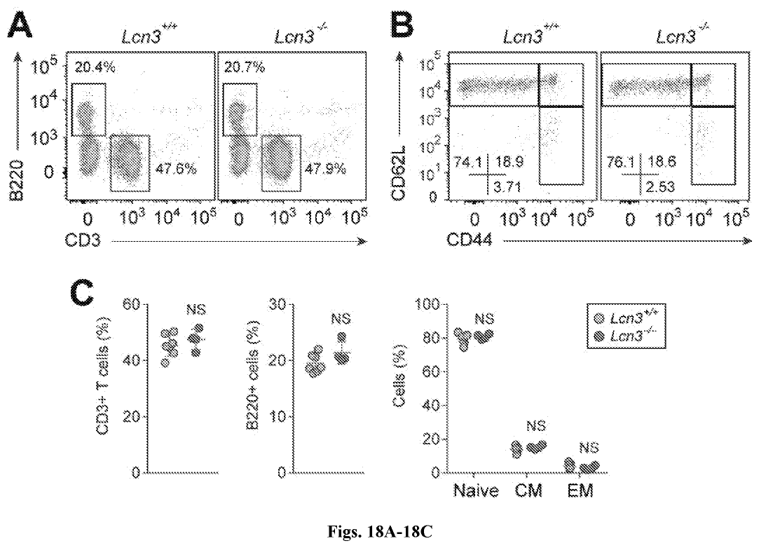

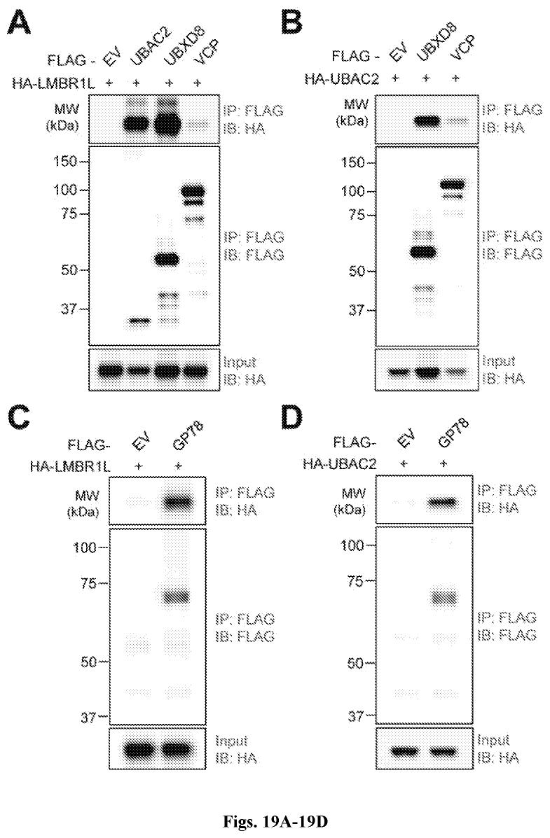

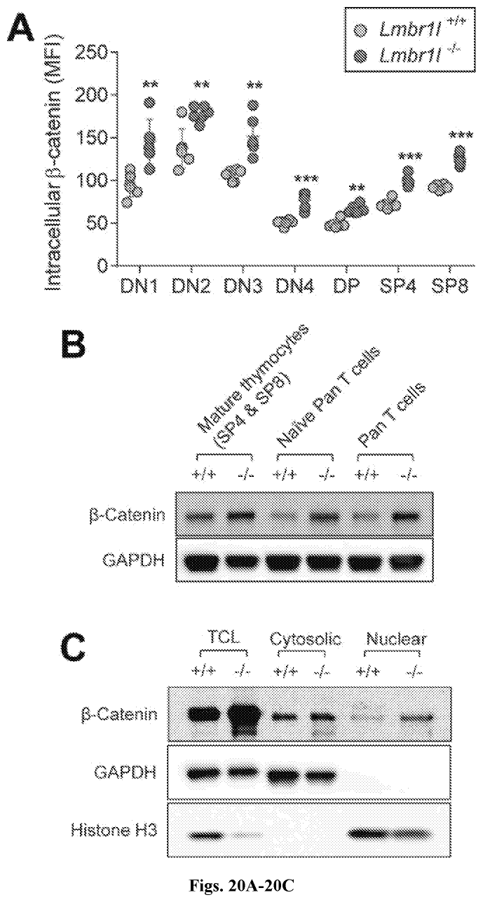

LMBR1L was first identified as a receptor for human lipocalin-1 (LCN1), an extracellular scavenger/carrier of lipophilic compounds that mediates ligand internalization and degradation (14-18). Later findings suggested that LMBR1L mediates internalization of bovine lipocalin β-lactoglobulin (BLG) (19), a major food-borne allergen in humans, and that LMBR1L interacts with uteroglobin (UG), which has anti-chemotactic properties (20). We generated mice carrying a targeted null allele of Lcn3, the mouse orthologue of human LCN1, and observed that LCN3-deficient mice were overtly normal and did not exhibit lymphocyte development defects. Thus, the function of LMBR1L in lymphopoiesis is independent of its interaction with LCN3 (FIGS. 18A-18C).

We sought to understand the immune function of LMBRL1 by identifying LMBR1L-interacting proteins using co-immunoprecipitation (co-IP) combined with mass spectrometry (MS) analysis. Among the 1,623 candidate proteins identified as putative LMBR1L interactors (Dataset S1), 25 proteins were >50-fold more abundant in the LMBR1L co-IP product relative to empty vector control (Table 1).

TABLE 1
LMBR1L interacting proteins identified by co-immunoprecipitation (IP) combined
with mass spectrometry (MS) analysis which were increased more than 50 fold or
Wnt components exclusively present in LMBR1L co-IP product relative to empty
vector control. Bold font: ERAD proteins; Italic font: Wnt related proteins.

| | | | | | Ratio |
|---|---|---|---|---|---|
| | | | Peptide | % | h-LMBR1L/ |
| Protein | Description | PSMs | Seqs. | Coverage | h-vector |
| Q8NBM4 | UBAC2 | 43 | 10 | 30.8 | 297.46 |
| Q9Y5M8 | SRPRB | 28 | 15 | 57.6 | 231.28 |
| Q9UHB9 | SRP68 | 9 | 7 | 16.8 | 197.80 |
| Q14697 | GANAB | 29 | 15 | 18.4 | 197.57 |
| F5GZJ1 | NCAPD2 | 21 | 15 | 14.7 | 168.60 |
| P55072 | VCP (TERA) | 78 | 34 | 44.7 | 120.13 |
| P04843 | RPN1 | 31 | 19 | 29.0 | 118.15 |
| Q14C86 | GAPVD1 | 16 | 8 | 8.1 | 110.42 |
| P53621 | COPA | 22 | 20 | 16.8 | 110.02 |
| O43678 | NDUFA2 | 4 | 3 | 41.4 | 107.10 |
| J3KR24 | IARS | 15 | 10 | 10.4 | 106.07 |
| B9A067 | IMMT | 23 | 20 | 30.3 | 99.95 |
| Q96SK2 | TMEM209 | 13 | 10 | 31.4 | 97.83 |
| Q14318-2 | FKBP8 | 14 | 10 | 26.9 | 87.63 |
| H3BS72 | PTPLAD1 | 19 | 10 | 15.7 | 87.63 |
| P57088 | TMEM33 | 11 | 6 | 21.9 | 87.04 |
| F8VZ44 | AAAS | 9 | 5 | 19.8 | 81.90 |
| Q07065 | CKAP4 | 30 | 17 | 35.9 | 79.54 |
| Q96CS3 | FAF2 | 26 | 11 | 27.2 | 71.19 |
| | (UBXDB8) | | | | |
| Q8WUM4 | PDCD6IP | 12 | 8 | 10.4 | 67.34 |
| P46977 | STT3A | 9 | 9 | 13.9 | 65.56 |
| E7EUU4 | EIF4G1 | 21 | 17 | 15.7 | 58.11 |
| Q9Y5V3 | MAGED1 | 16 | 11 | 19.3 | 58.07 |
| Q9UKV5 | AMFR | 25 | 12 | 25.2 | 51.68 |
| | (GP78) | | | | |
| Q93008 | USP9X | 19 | 13 | 8.3 | 51.59 |
| Q9ULT6 | ZNRF3 | 9 | 6 | 11.2 | h-LMBR1L only |
| F5H7J9 | LRP6 | 4 | 3 | 3.2 | h-LMBR1L only |
| B4DGU4 | CTNNB1 | 4 | 3 | 4.7 | h-LMBR1L only |
| P49840 | GSK3A | 3 | 4 | 13.9 | h-LMBR1L only |
| P49841 | GSK3B | 1 | 3 | 7.1 | h-LMBR1L only |

Four of the proteins were essential components of the ERAD pathway, including ubiquitin associated domain containing 2 (UBAC2; elevated 297-fold), transitional endoplasmic reticulum ATPase (TERA known as VCP; elevated 120-fold), UBX domain-containing protein 8 (UBXD8, known as FAF2; elevated 71-fold) (21), and glycoprotein 78 (GP78; known as AMFR; elevated 51-fold). We also identified numerous components of the Wnt/β-catenin signaling pathway that were among 764 proteins found exclusively in the LMBR1L co-IP, including zinc and ring finger 3 (ZNRF3), low-density lipoprotein receptor-related protein 6 (LRP6), β-catenin, glycogen synthase kinase-3a (GSK3a), and GSK3β. We also performed protein microarray analysis as a second unbiased approach to identify LMBR1L-interacting proteins. GSK-3β ranked eighth out of 9,483 human proteins for binding affinity to LMBR1L (FIG. 4A, Dataset S2). LMBR1L showed binding affinity for casein kinase 1 (CK1) isoforms including CK1α, γ, δ, and ε, as well as for β-catenin. To confirm the interactions between LMBR1L and components of the Wnt/β-catenin signaling or ERAD pathways, HEK293T cells were co-transfected with HA-tagged LMBR1L and FLAG-tagged GSK-3β, β-catenin, ZNRF3, ring finger 43 (RNF43, a homologue of ZNRF3 with redundant function in Wnt receptor processing), FZD6, LRP6, or DVL2. LMBR1L co-immunoprecipitated with each of the FLAG-tagged proteins (FIG. 4B). Furthermore, co-IP and immunoblot analysis confirmed that LMBR1L interacts with each of the ERAD components including UBAC2, UBXD8, VCP, and GP78 (FIGS. 19A-19D). Thus, LMBR1L may be a critical component of the Wnt/β-catenin and ERAD signaling pathways.

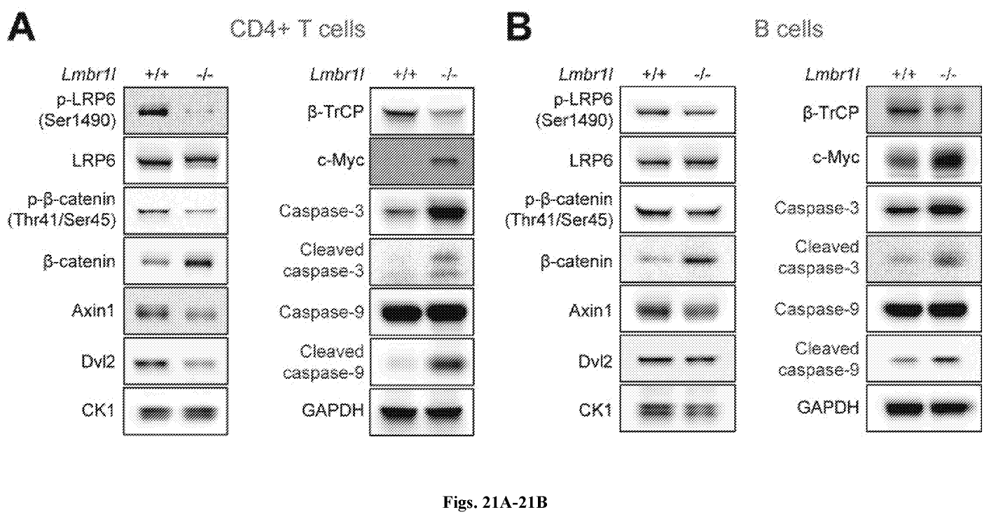

To determine the relationship between Wnt/β-catenin signaling and LMBR1L, we examined Wnt/β-catenin signaling in CD8+ T cells from Lmbr1l−/− and wild-type mice. A key regulatory step in the Wnt/β-catenin signaling pathway involves the phosphorylation, ubiquitination, and subsequent degradation of the Wnt downstream effector protein, β-catenin (22). LMBR1L deficiency resulted in β-catenin accumulation with concomitant decreased levels of phosphorylated-O-catenin relative to those in wild-type cells (FIG. 4C). The β-catenin accumulation was observed in developing thymocytes (DN1-4, DP, SP4, SP8; FIGS. 20A, 20B) as well as nave and mature T cells in the periphery (FIG. 20B). To determine whether there were changes in the localization of β-catenin, nuclear and cytosolic extracts were isolated from Lmbr1l−/− CD8+ T cells. Immunoblotting revealed increased β-catenin levels in the nuclear fraction of Lmbr1l−/− CD8+ T cells compared to wild-type cells (FIG. 20C). Tonic β-catenin inactivation requires phosphorylation of β-catenin by GSK-3α/β and CK1 within an intact destruction complex composed of scaffolding proteins Axin1 and DVL2, followed by ubiquitination mediated by E3 ubiquitin ligase β-TrCP (5, 22). Lmbr1l−/− CD8+ T cells showed decreased total GSK-3α/β and CK1 levels with concomitant increased levels of the inactive form of GSK-3β (phosphorylated-GSK-3β; FIG. 4C). Additionally, Axin1, DVL2, and β-TrCP levels were reduced in Lmbr1l−/− CD8+ T cells compared to wild-type cells (FIG. 4C). Nuclear accumulation of β-catenin upon Wnt activation facilitates upregulation of its target genes, including CD44 and c-Myc. Consistent with the increased β-catenin levels in the nuclear fraction of Lmbr1l−/− CD8+ T cells, we found increased c-Myc expression in total cell lysates (FIG. 4C). c-Myc-induced apoptosis is p53-dependent. The anti-apoptotic cell cycle arrest protein p21 is a target of p53, and is transcriptionally repressed by c-Myc (23). LMBR1L deficiency increased p53 expression, suppressed p21, and increased caspase-3 and -9 cleavage (FIG. 4C). LMBR1L deficiency produced similar effects in CD4+T and B cells (FIGS. 21A-21B).

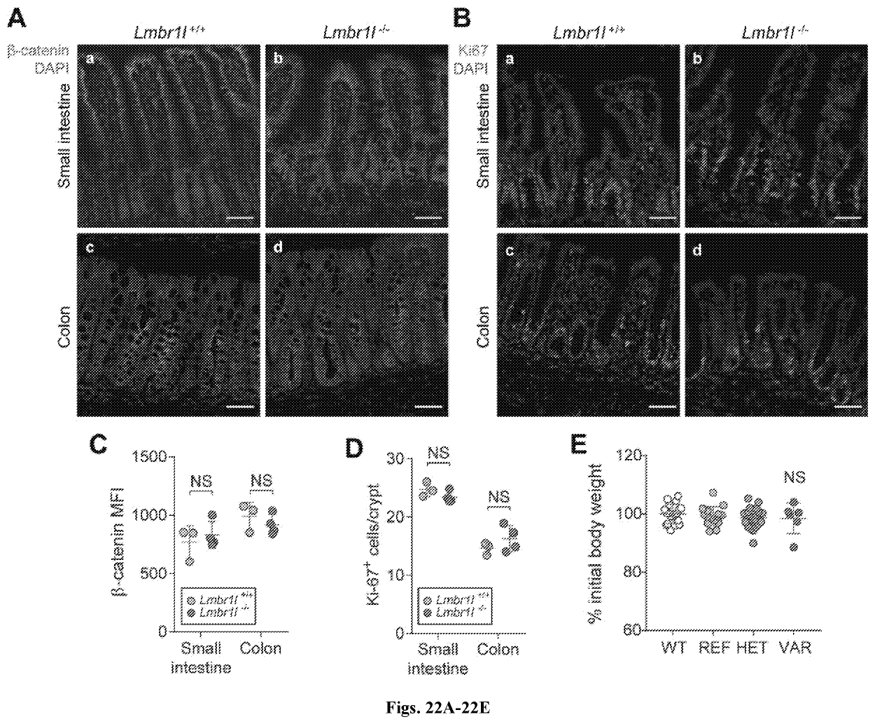

Aberrant Wnt activation in the intestinal epithelium results in adenoma formation and colon cancer (9). However, Lmbr1l−/− intestinal epithelium did not show β-catenin accumulation (FIGS. 22A, 22C), marked expansion of crypts determined by Ki-67 staining (FIGS. 22B, 22D), or intestinal homeostatic abnormalities after oral administration of dextran sodium sulfate (DSS; FIG. 22E). Consistent with the absence of Lmbr1l mRNA expression in LGR5+ intestinal stem cells (24), our findings suggest that other system(s) for regulating β-catenin activity are redundant with LMBR1L in the gut cell environment. These results establish LMBR1L as a lymphocyte-specific negative regulator of Wnt/β-catenin signaling.

The LMBR1LGP78UBAC2 Complex Regulates the Maturation of Wnt Receptors within the ER and Stabilizes GSK-3β

Wnt proteins bind to a receptor complex of two molecules, FZD and LRP6 (5). Our findings suggest that LMBR1L acts as a negative regulator of the Wnt pathway. Therefore, we examined whether LMBR1L could regulate Wnt co-receptor expression and/or stability of the destruction complex. An increased level of the mature (glycosylated) form of FZD6 was detected in the membrane fraction of Lmbr1l−/− CD8+ T cells relative to wild-type cells (FIG. 5A). Both mature and immature forms of FZD6 and LRP6 were increased in total cell lysates (TCLs) of Lmbr1l−/− CD8+ T cells compared to wild-type CD8+ T cells (FIG. 5A).

ZNRF3 and RNF43 are negative regulators of the Wnt pathway. ZNRF3 and RNF43 selectively ubiquitinate lysines in the cytoplasmic loops of FZD, which targets FZD for degradation at the plasma membrane (8). In addition, DVL proteins act as an intermediary for ZNRF3/RNF43-mediated ubiquitination and degradation of FZD (10). We found that ZNRF3/RNF43 levels were altered in the membrane fraction of Lmbr1−/− CD8+ T cells compared to levels in wild-type cells. In the TCLs, ZNRF3 levels were unchanged, whereas RNF43 levels were slightly increased (FIG. 5A).

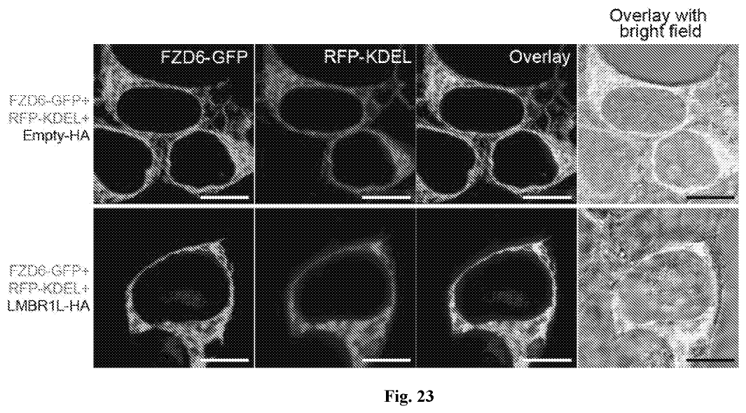

UBAC2 is a core component of the GP78 ubiquitin ligase complex expressed on the ER membrane. UBAC2 physically interacts with and adds poly-UB chains to UBXD8, a protein involved in substrate extraction during ERAD (21, 25). We hypothesized that the interaction of LMBR1L with UBAC2, GP78, and UBXD8 might regulate the activity of the GP78 ubiquitin ligase complex towards FZD and/or LRP6. Transient co-transfection of HEK293T cells with FLAG-tagged FZD6 and HA-tagged LMBR1L or UBAC2 resulted in decreased total levels of the mature FZD6 (FIG. 5B). Co-expression of FLAG-tagged FZD6 and HA-tagged GP78 strongly decreased both the mature and immature form of FZD6. In contrast to LMBR1L, UBAC2 and GP78 strongly promoted ubiquitination of FZD6 (FIG. 5B). In addition, whereas GFP-tagged FZD6 localized to both plasma membrane and ER in HEK293T cells, co-expression of LMBR1L with FZD6-GFP altered the localization of FZD6-GFP, causing it to accumulate in the ER and inhibiting its expression on the plasma membrane (FIG. 23). ER stress was observed in Lmbr1l−/− CD8+ T cells as indicated by increased expression of binding immunoglobulin protein (BiP) and glucose-regulated protein 94 (GRP94) compared to wild-type cells (FIG. 5A). We also found that LMBR1L expression preferentially decreased mature LRP6 whereas UBAC2 decreased both mature and immature LRP6 (FIG. 5C). LMBR1L, for which no functional domain has previously been reported, is known to localize at the plasma membrane (17, 18). However, our data suggest that LMBR1L may function as a core component of the GP78 UBAC2 ubiquitin ligase complex, and that LMBR1L-mediated maturation of Wnt co-receptors may be regulated within the ER.

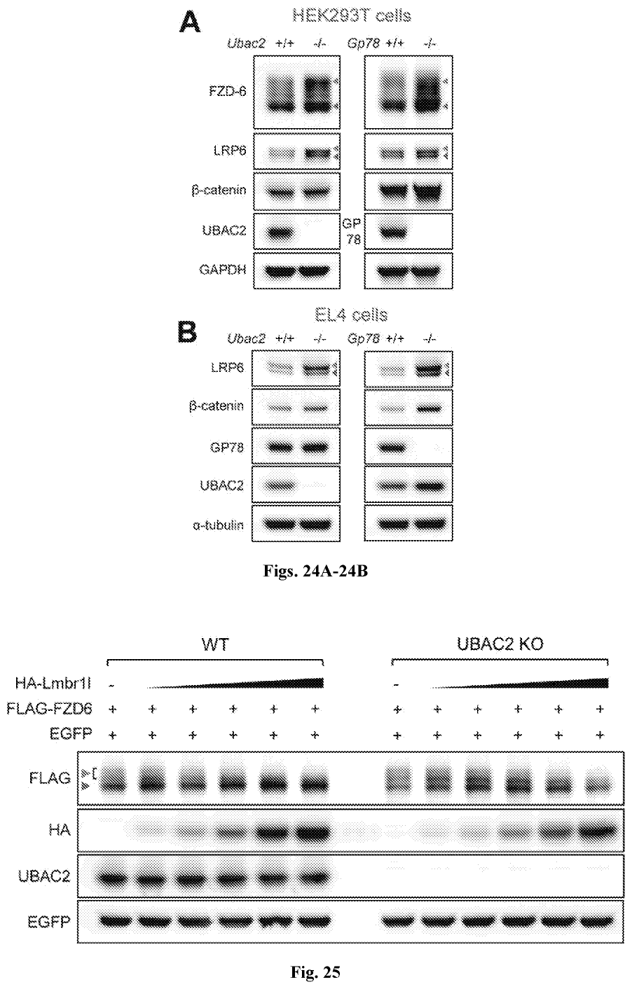

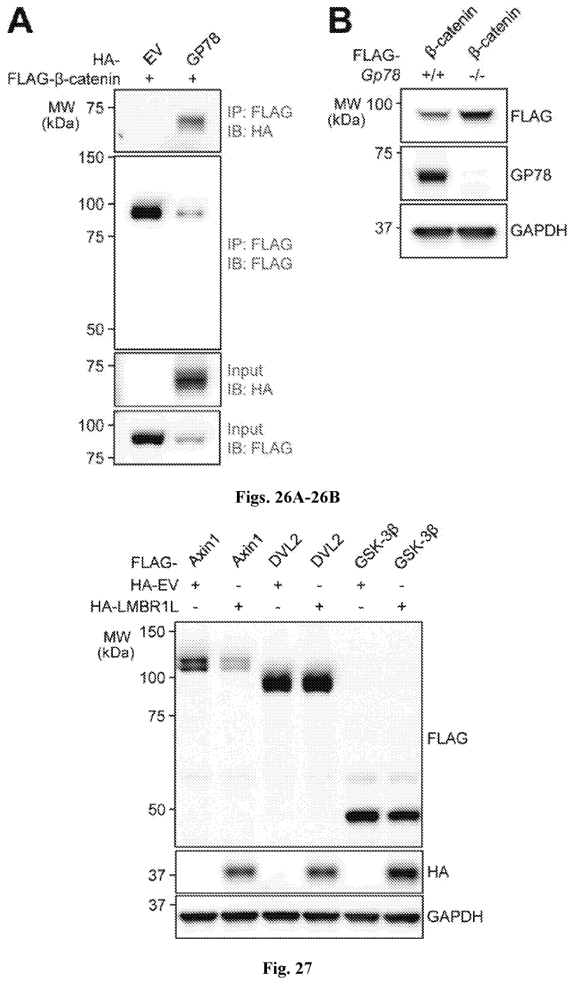

To test this hypothesis, we generated a CRISPR-based knock-in of a FLAG-tag appended to the C-terminus of endogenous LMBR1L protein in HEK293T cells. Most LMBR1L-FLAG was expressed in the ER of these cells, and only a small fraction was localized to the plasma membrane (FIG. 5D). We also knocked out Ubac2 or Gp78 in HEK293T cells (FIG. 24A) and the mouse T cell line EL4 (FIG. 24B) using the CRISPR/Cas9 system. Increased FZD6 and LRP6 were detected in both the Ubac2−/− and Gp78−/− cells relative to the parental HEK293T or EL4 cells (FIG. 24A, 24B). Similar to LMBR1L deficiency in primary CD8+ T cells, GP78 deficiency in HEK293T or EL4 cells also resulted in β-catenin accumulation (FIG. 24A, 24B). Furthermore, CRISPR/Cas9-targeted Gp78 knockout mice were generated and used to confirm that GP78 deficiency in primary CD8⁺ T cells results in increased FZD6 and LRP6 expression as well as β-catenin accumulation (FIG. 5E). We also examined the effect of UBAC2 on FZD6 maturation mediated by LMBR1L after transient transfection of FLAG-tagged FZD6, HA-tagged LMBR1L, and EGFP in Ubac2−/− or parental HEK293T cells. Increasing amounts of LMBR1L significantly reduced the amount of mature FZD6 and increased the amount of immature FZD6 without affecting EGFP expression in wild-type HEK293T cells (FIG. 25). However, increasing amounts of LMBR1L in Ubac2−/− cells failed to inhibit FZD6 maturation as efficiently as in wild-type cells (FIG. 25). In addition, the preferential inhibition of mature LRP6 by LMBR1L observed in wild-type cells was partially rescued in Gp78−/− cells, and total expression of LRP6 was notably higher (FIG. 5F). Similarly, transient co-transfection of HEK293T cells with FLAG-tagged β-catenin and HA-tagged LMBR1L, UBAC2, or GP78 resulted in decreased total levels of β-catenin compared to the empty vector control (FIG. 5G). Using co-IP, we confirmed a physical interaction between GP78 and β-catenin (FIG. 26A). Co-expression of FLAG-tagged β-catenin and HA-tagged GP78 strongly promoted the ubiquitination of β-catenin (FIG. 5G). Conversely, increased β-catenin expression was observed in Gp78−/− cells compared to parental HEK293T cells after transient transfection of FLAG-tagged β-catenin (FIG. 26B). Thus, the LMBR1LGP78UBAC2 complex appears to prevent maturation of FZD6 and the Wnt co-receptor LRP6 within the ER of lymphocytes. Furthermore, the LMBR1LGP78UBAC2 complex may regulate the ubiquitination and degradation of (3-catenin.

Another striking difference observed in Lmbr1l−/− T cells was that several components of the destruction complex were expressed at lower levels than in wild-type cells, including scaffolding protein Axin1, DVL2, kinases GSK-3α/β and CK1, and the E3 ligase β-TrCP (FIG. 4C). Furthermore, Lmbr1l−/− T cells showed the decreased expression of phosphorylated-β-catenin and phosphorylated-LRP6 (FIG. 4C and FIG. 5A, respectively), increased phosphorylated-GSK-3β (FIG. 4C), and the activation of kinases such as Akt and p70S6K (FIG. 3B) which inactivates GSK-3β by phosphorylation. Thus, we hypothesized that the LMBR1L GP78UBAC2 complex may regulate the stability of destruction complex components such as GSK-3β, which has both inhibitory and stimulatory roles in Wnt/β-catenin signaling by phosphorylating β-catenin and LRP6, respectively (26). Transient co-transfection of HEK293T cells with FLAG-tagged Axin1, DVL2, or GSK-3β and HA-tagged LMBR1L or empty vector resulted in decreased total levels of FLAG-tagged Axin1 protein in the presence of HA-LMBR1L compared to empty vector control. However, LMBR1L had no effect on DVL2 or GSK-3β expression (FIG. 27), nor on the level of phosphorylated GSK-3β (FIG. 6A). To measure the effect of LMBR1L on the half-life of GSK-3β, HEK293T cells were transfected with FLAG-tagged GSK-3β and HA-tagged LMBR1L or empty vector. Fourteen hours after transfection, cells were treated with the translation inhibitor cycloheximide (CHX) and harvested at various times post-treatment. In the presence of LMBR1L, no detectable decrease of GSK-3β was observed up to 4 h after CHX treatment, suggesting that LMBR1L stabilizes GSK-3β (FIG. 6B).

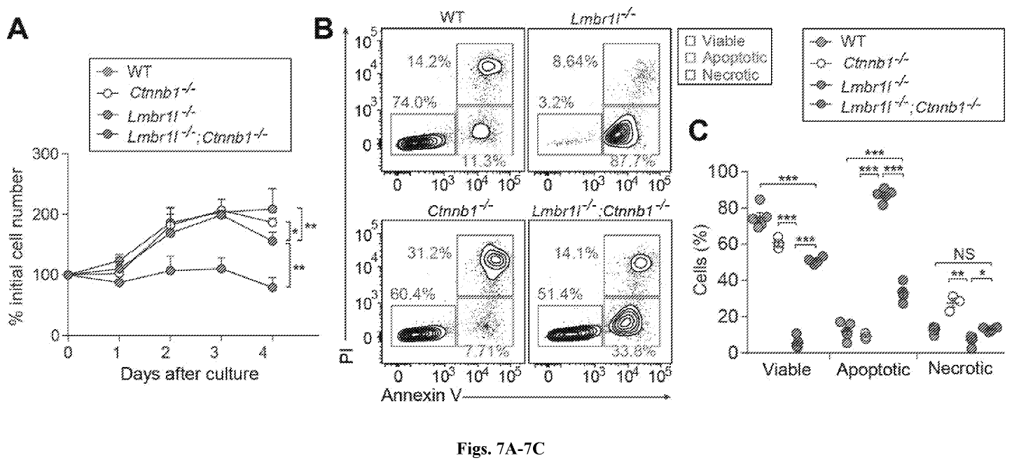

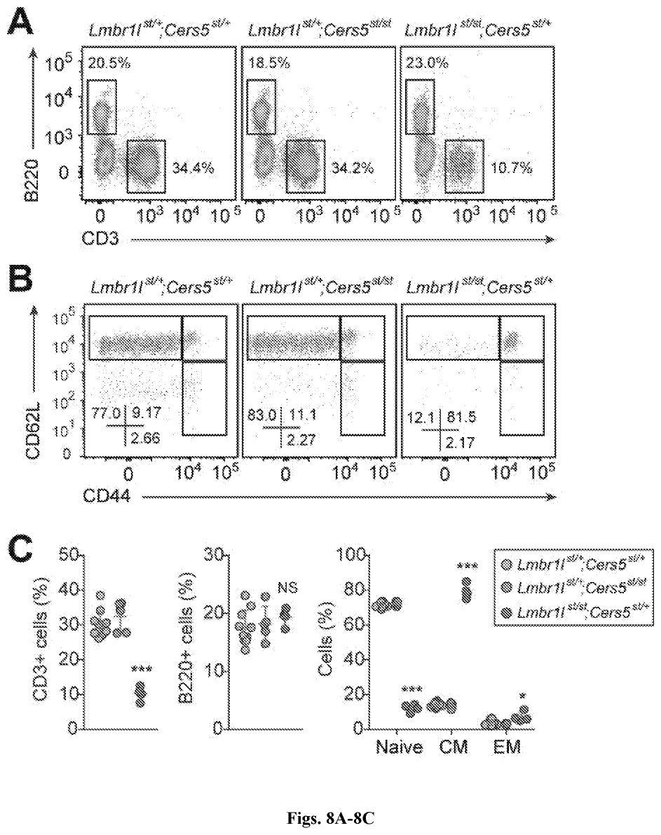

Cumulative evidence suggests that LMBR1L serves as a negative regulator of Wnt/β-catenin signaling. To test whether the observed phenotypes depend on the pathway, we knocked out Lmbr1l, β-catenin (Ctnnb1), or Lmbr1l and Ctnnb1 in EL4 cells using the CRISPR/Cas9 system. Similar to the phenotype observed in primary Lmbr1l−/− CD8+ T cells, Lmbr1l−/− EL4 cells showed severe defects in proliferation even under normal culture conditions (FIG. 7A). Annexin V and PI staining showed that the majority of the Lmbr1l−/− EL4 cells were apoptotic (FIG. 7B: top right, 7C). An increased frequency of necrotic cells was detected among Ctnnb1−/− EL4 cells compared to parental wild-type EL4 cells (FIG. 7B: bottom left, 7C); however, their growth was normal (FIG. 7A). Deletion of Ctnnb1 in Lmbr1l−/− EL4 cells substantially restored proliferative potential and decreased apoptosis compared to Lmbr1l−/− EL4 cells (FIG. 7A, B: bottom right, 7C); however, proliferation and apoptosis did not reach levels observed in parental wild-type Ctnnb1−/− EL4 cells (FIG. 7A, 7B). These results provide genetic evidence that β-catenin is downstream of LMBR1L in a mouse T lymphocyte transformed cell line and suggest that the observed phenotypes in LMBR1L deficient T cells are largely dependent on Wnt/β-catenin signaling.

### LMBR1L Deficiency Inhibits Autoantibody Production and B Cell Survival in Mice

The production of autoantibodies (dsDNA-specific IgG) is the most specific and sensitive indication for systemic lupus erythematosus compared to other autoimmune diseases. To gain insights into the regulation of LMBR1L in autoimmune disease, Lmbr1l^{-/-} mice were crossed to Tg(BCL2)22Wehi/J mouse strain (hereafter Bcl2-Tg) which express a transgene containing human B-cell lymphoma 2 (BCL2) cDNA that is restricted to B cell lineage with no T cell expression. It was known that expression of the human BCL2 transgene in B cells enhances cell survival and promotes autoantibody production.

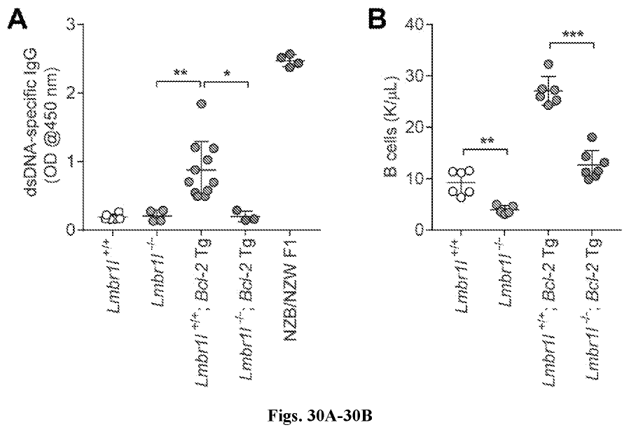

Here we found that Linbr1l^{-/-}; Bcl2-Tg mice have significantly lower dsDNA-specific IgG levels in serum compared to those from Linbr1l^{+/+}; Bcl2-Tg mice (FIG. 30A). Sera from 6 months old NZB/NZW F1 hybrid females served as positive control for dsDNA-specific antibody measurement. This result suggests that LMBR1L deficiency inhibits autoimmune response. Furthermore, quantitation of peripheral blood B cells in Linbr1l^{+/+}; Bcl2-Tg, and Lmbr1l^{-/-}; Bcl2-Tg mice revealed that LMBR1L deficiency significantly inhibits B cell survival in mice (FIG. 30B).

### Concluding Remarks

Our findings demonstrate the existence of a pathway that regulates Wnt/β-catenin signaling in lymphocytes. The exaggerated apoptosis of T cells that results in lymphopenia in LMBR1L-deficient mice stems from the aberrant activation of Wnt/β-catenin signaling. In the absence of LMBR1L, the expression of mature forms of Wnt co-receptors and phosphorylated GSK-3β were highly upregulated, whereas the expression of multiple destruction complex proteins was reduced. These alterations contributed to the accumulation of β-catenin, which enters the nucleus and promotes the transcription of target genes such as Myc, Trp53, and Cd44. This signal transduction cascade favors apoptosis in an intrinsic and extrinsic caspase cascade-dependent manner.

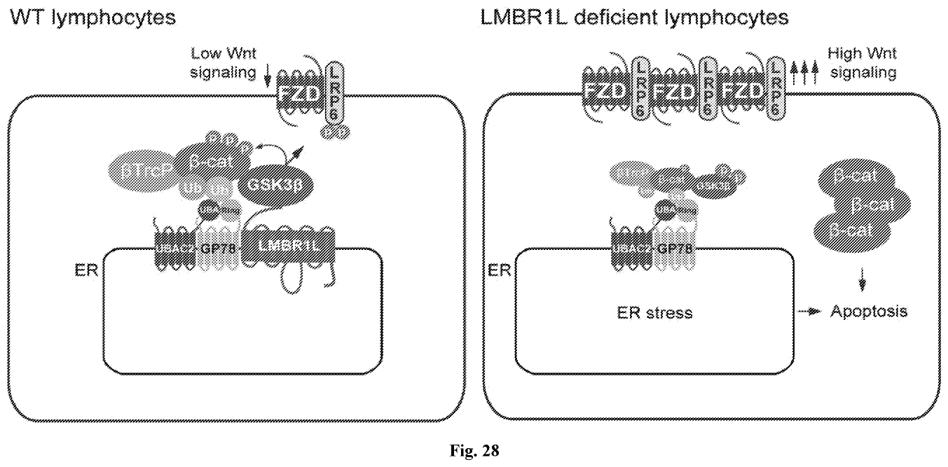

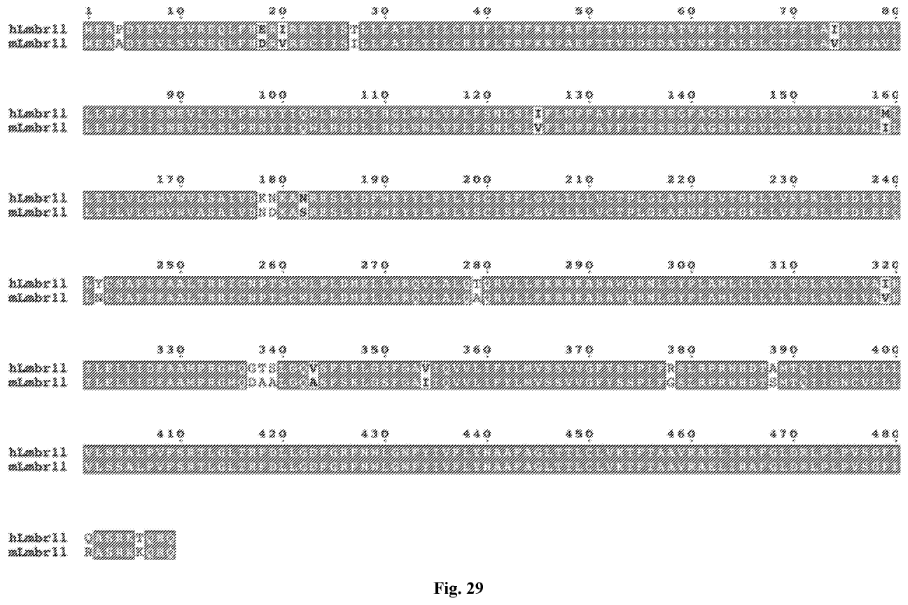

We report herein a second “destruction complex” in the ER, comprising LMBR1L, GP78, and UBAC2, which controls Wnt signaling activity in lymphocytes by regulating Wnt receptor availability independent of ligand binding (FIG. 28). Furthermore, LMBR1L supports the expression and/or stabilization of the canonical destruction complex including GSK-3β that is necessary for degradation of β-catenin and activation of LRP6. Because human and mouse LMBR1L orthologues share 96% identity (FIG. 29), we believe the same mechanism operates in human lymphoid cells and their progenitors. LMBR1L deficiency may be considered as a possible etiology in unexplained pan-lymphoid immunodeficiency disorders.

### Materials and Methods

### Mice

Eight-to-ten-week old pure C57BL/6J background males purchased from The Jackson Laboratory were mutagenized with N-ethyl-N-nitrosourea (ENU) as described previously (27). Mutagenized G0 males were bred to C57BL/6J females, and the resulting G1 males were crossed to C57BL/6J females to produce G2 mice. G2 females were backcrossed to their G1 sires to yield G3 mice, which were screened for phenotypes. Whole-exome sequencing and mapping were performed as described (11). C57BL/6.SJL (CD45.1), Rag2^{-/-}, Tnf-α^{-/-}, Casp3^{-/-}, Fas^{lpr}, B2m^{tm1Unc} (B2 m^{-/-}) and Tg(TcraTcrb)1100Mjb (OT-I) transgenic mice were purchased from The Jackson Laboratory. CD45.1; Lmbr1l^{-/-}, Tnf-α^{-/-}, Lmbr1l^{-/-}, Casp2^{-/-}, Lmbr1l^{-/-}; Fas^{lpr/lpr}, Lmbr1l^{-/-}; OT-I mice were generated by intercrossing mouse strains. Mice were housed in specific pathogen-free conditions at the University of Texas Southwestern Medical Center and all experimental procedures were performed in accordance with institutionally approved protocols.

### Bone Marrow Chimeras

Recipient mice were lethally irradiated with 13 Gy via gamma radiation (X-RAD 320, Precision X-ray Inc.). The mice were given an intravenous injection of 5×10⁶ bone marrow cells derived from the tibia and femurs of the respective donors. For 4 weeks post-engraftment, mice were maintained on antibiotics. Twelve weeks after bone marrow engraftment, the chimeras were euthanized to assess immune cell development in bone marrow, thymus, and spleen by flow cytometry. Chimerism was assessed using congenic CD45 markers.

### Flow Cytometry

Bone marrow cells, thymocytes, splenocytes, or peripheral blood cells were isolated, and red blood cell (RBC) lysis buffer was added to remove RBCs. Cells were stained at a 1:200 dilution with 15 mouse fluorochrome-conjugated monoclonal antibodies specific for the following murine cell surface markers encompassing the major immune lineages: B220, CD3ε, CD4, CD5, CD8α, CD11b, CD11c, CD19, CD43, CD44, CD62L, F4/80, IgD, IgM, and NK1.1 in the presence of anti-mouse CD16/32 antibody for 1 h at 4° C. After staining, cells were washed twice in PBS and analyzed by flow cytometry.

To stain the hematopoietic progenitor compartment, bone marrow was isolated and stained with Alexa Fluor 700-conjugated lineage markers (B220, CD3, CD11b, Ly-6G/6C, and Ter-119), CD16/32, CD34, CD135, c-Kit, IL-7Rα, and Sca-1 for 1 h at 4° C. After staining, cells were washed twice in PBS and analyzed by flow cytometry.

PE-conjugated K^{b}/SIINFEKL tetramer, a reagent specific for the ovalbumin epitope peptide SIINFEKL presented by H-2K^{b} (MHC Tetramer Core at Baylor College of Medicine) was used to detect antigen-specific CD8⁺ T cell responses and memory CD8⁺ T cell formation in mice immunized with aluminum-hydroxide precipitated ovalbumin.

To detect intracellular β-catenin, thymi were homogenized to generate a single-cell suspension and surface stained for CD3, CD25, and CD44. Cells were then permeabilized using BD Cytofix/Cytoperm Kit followed by intracellular β-catenin staining. Data were acquired on an LSRFortessa cell analyzer (BD Bioscience) and analyzed with FlowJo software (Treestar).

### Immunization

Twelve-to-sixteen-week-old G3 mice or Lmbr1l^{-/-}, Cers5^{-/-}, and wild-type littermates were immunized (i.m.) with T cell-dependent antigen (TD) aluminum hydroxide-precipitated ovalbumin (OVA/alum; 200 μg; Invivogen) on day 0. Fourteen days after OVA/alum immunization, blood was collected in MiniCollect Tubes (Mercedes Medical) and centrifuged at 1,500×g to separate the serum for ELISA analysis. Three days after bleeding, mice were immunized with another TD antigen, rSFV-βGal (2×10⁶ IU; (28)) on day 0 and the T cell-independent antigen (TI) NP₅₀-AECM-Ficoll (50 μg; Biosearch Technologies) on day 8 (i.p.) as previously described (29). Six days after NP₅₀-AECM-Ficoll immunization, blood was collected for ELISA analysis.

### In Vivo CTL and NK Cytotoxicity

Cytolytic CD8⁺ T cell effector function was determined by a standard in vivo cytotoxic T lymphocyte (CTL) assay. Briefly, splenocytes were isolated from naïve mice and divided in half According to established methods (30), half were stained with 5 μM CFSE (CFSE^{hi}), and half were labeled with 0.5 μM CFSE)(CFSE^{lo}. CFSE^{hi} cells were pulsed with 5 μM ICPMYARV peptide, which carries E. coli β-galactosidase MHC I epitope for mice with the H-2b haplotype (New England Peptide; (31). CFSE^{lo} cells were not stimulated. CFSE^{hi} and CFSE^{lo} cells were mixed (1:1) and 2×10⁶ cells were administered to naïve mice and mice immunized with rSFV-βgal through retro-orbital injection. Blood was collected 24 h after adoptive transfer, and CFSE intensities from each population were assessed by flow cytometry. Lysis of target (CFSE^{hi}) cells was calculated as: % lysis=[1 (ratio_{control mice}/ratio_{vaccinated mice})]×100; ratio=percent CFSE^{lo}/percent CFSE^{hi}.

To measure NK cell-mediated killing, splenocytes from control C57BL/6J (0.5 μM Violet; Violet^{lo}) and B2 m^{-/-} mice (5 μM Violet; Violet^{hi}) were stained with CellTrace Violet. Equal numbers of Violet^{hi} and Violet^{lo} cells were transferred by retro-orbital injection. Twenty-four hours after transfer, blood was collected and Violet intensity from each population was assessed by flow cytometry. % lysis=[1 (target cells/control cells)/(target cells/control cells in B2 m^{-/-})]×100.

### MCMV Challenge

Mice were infected with MCMV (Smith strain; 1.5×10⁵ pfu/20 g of body weight) by intraperitoneal injection as described previously (32). Mice were euthanized 5 days after MCMV challenge to determine viral loads. Total DNA extracted from individual mouse spleen was used to measure copy numbers of MCMV immediate-early 1 (IE1) gene and control DNA sequence (β-actin). The viral titer is represented as the copy number ratio of MCMV IE1 to β-actin.

### In Vivo T Cell Activation

Splenic CD45.2⁺ OT-I and Linbr1^{-/-}; OT-1 T cells were purified using the EasySep™ Mouse CD8⁺ T Cell Isolation Kit (StemCell Technologies). Purities were over 95% in all experiments as tested by flow cytometry. Cells were labeled with 5 μM CellTrace Far Red (CD45.2⁺ OT-I) or 5 μM CellTrace Violet (Lmbr1^{-/-}; OT-1), and equal number of stained cells (2×10⁶) cells were injected by retro-orbital route into wild-type CD45.1⁺ mice. The next day, recipients were injected with either 100 μg of soluble OVA in 200 μl of PBS or 200 μl of sterile PBS as a control. Antigen (OVA)-specific T cell activation was analyzed based on Far Red or Violet intensity of dividing OT-I cells after 48 h and 72 h.

To assess the proliferative capacity of T cells in response to homeostatic proliferation signals, splenic pan T cells or mature SP thymocytes (CD24⁻) were isolated by using the EasySep™ Mouse Pan T Cell Isolation Kit (StemCell Technologies) or Dynal negative selection using biotinylated anti-CD24 mAb M1/69 (eBioscience), respectively. Pan T or mature CD24⁻ thymocytes isolated from Lmbr1l^{-/-}, Lmbr1l^{st/st} or wild-type littermates were stained with 5 μM CellTrace Violet or CellTrace Far Red, respectively. A 1:1 or 10:1 mix of labeled Lmbr1l^{-/-} or wild-type cells was transferred into C45.1⁺ mice that had been sublethally irradiated (8 Gy) 6 h earlier or into unirradiated controls. Four or seven days after adoptive transfer, splenocytes were prepared, surface stained for CD45.1, CD45.2, together with CD3, CD4, and CD8 and then analyzed by flow cytometry for Far Red or Violet dye dilution.

### Detection of Apoptosis

Annexin V/PI labeling and detection was performed with the FITC-Annexin V Apoptosis Detection Kit I (BD Bioscience) according to manufacturer's instructions.

### Mass Spectrometry Analysis

Co-immunoprecipitation and mass spectrometry were performed to identify Lmbr1l-interacting proteins as described below. Transfection was performed in HEK293T cells (ATCC) using Lipofectamine 2000 reagent (Life technologies) with plasmid encoding Flag tagged-human Lmbr1l or empty vector control. Forty-eight hours after transfection, cells were harvested in NP-40 lysis buffer for 45 min at 4° C. Immunoprecipitation was performed using anti-FLAG M2 affinity gel (Sigma) for 2 h at 4° C. and beads were washed six times in NP-40 lysis buffer. The proteins were eluted with SDS sample buffer and heated at 95° C. for 10 min. Lysates were loaded onto 12% (wt/vol) SDS-PAGE gel and run 1 cm into the separation gel. The gel was stained with Coomassie blue (Thermo Fisher) and whole stained lanes were subjected to mass spectrometry analysis (LCMS/MS) as described previously (33).

### Protein Array

The ProtoArray Human Protein Microarray V5.1 (Invitrogen) was used to identify human Lmbr1l-interacting proteins according to manufacturer's instructions. Briefly, Flag (N-terminus)- and V5 (C-terminus)-tagged recombinant human Lmbr1l protein was expressed in HEK293T cells by transfection and purified with anti-FLAG M2 affinity gel (Sigma). The presence of the Flag and V5 tag on the protein was confirmed by standard immunoblot.

Purified recombinant Flag-human LMBR1L-V5 was used to probe a Human V5.1 ProtoArray (Invitrogen) at a final concentration of 50 μg/ml. Binding of the recombinant protein on the array was detected with streptavidin Alexa Fluor 647 at a 1:1,000 dilution in Protoarray blocking buffer (Invitrogen). The array was scanned using a GeneArray 4000B scanner (Molecular Devices) at 635 nm. Results were saved as a multi-TIFF file and analyzed using Genepix Prospector software, version 7.

### Isolation of Plasma Membrane or Endoplasmic Reticulum

Proteins from the plasma membrane or endoplasmic reticulum were isolated using the Pierce Cell Surface Protein Isolation Kit (Thermo Fisher) or Endoplasmic Reticulum Enrichment Extraction Kit (Novus Biologicals), respectively, according to manufacturer's instructions.

### Statistical Analysis

The statistical significance of differences between groups was analyzed using GraphPad Prism by performing the indicated statistical tests. Differences in the raw values among groups were considered statistically significant when P<0.05. P-values are denoted by *P<0.05; ** P<0.01; ***P<0.001; NS, not significant with P>0.05.

### REFERENCES

1. S. Verbeek et al., An HMG-box-containing T-cell factor required for thymocyte differentiation. Nature 374, 70-74 (1995).
2. T. Reya et al., Wnt signaling regulates B lymphocyte proliferation through a LEF-1 dependent mechanism. Immunity 13, 15-24 (2000).
3. T. Reya et al., A role for Wnt signalling in self-renewal of haematopoietic stem cells. Nature 423, 409-414 (2003).
4. F. J. Staal, T. C. Luis, M. M. Tiemessen, WNT signalling in the immune system: WNT is spreading its wings. Nat Rev Immuno/8, 581-593 (2008).
5. R. Nusse, H. Clevers, Wnt/beta-Catenin Signaling, Disease, and Emerging Therapeutic Modalities. Cell 169, 985-999 (2017).
6. C. Wong, C. Chen, Q. Wu, Y. Liu, P. Zheng, A critical role for the regulated wnt-myc pathway in naive T cell survival. J Immunol 194, 158-167 (2015).
7. X. Jiang, F. Cong, Novel Regulation of Wnt Signaling at the Proximal Membrane Level. Trends Biochem Sci 41, 773-783 (2016).
8. H. X. Hao et al., ZNRF3 promotes Wnt receptor turnover in an R-spondin-sensitive manner. Nature 485, 195-200 (2012).
9. B. K. Koo et al., Tumour suppressor RNF43 is a stem-cell E3 ligase that induces endocytosis of Wnt receptors. Nature 488, 665-669 (2012).
10. X. Jiang, O. Charlat, R. Zamponi, Y. Yang, F. Cong, Dishevelled promotes Wnt receptor degradation through recruitment of ZNRF3/RNF43 E3 ubiquitin ligases. Mol Cell 58, 522-533 (2015).
11. T. Wang et al., Real-time resolution of point mutations that cause phenovariance in mice. Proc Natl Acad Sci USA 112, E440-449 (2015).
12. T. C. Luis et al., Canonical wnt signaling regulates hematopoiesis in a dosage-dependent fashion. Cell Stem Cell 9, 345-356 (2011).
13. D. M. Zhao et al., Constitutive activation of Wnt signaling favors generation of memory CD8 T cells. J Immunol 184, 1191-1199 (2010).
14. B. J. Glasgow, A. R. Abduragimov, Z. T. Farahbakhsh, K. F. Faull, W. L. Hubbell, Tear lipocalins bind a broad array of lipid ligands. Curr Eye Res 14, 363-372 (1995).
15. R. W. Hesselink, J. B. Findlay, Expression, characterization and ligand specificity of lipocalin-1 interacting membrane receptor (LIMR). Mol Membr Biol 30, 327-337 (2013).
16. B. Redl, P. Holzfeind, F. Lottspeich, cDNA cloning and sequencing reveals human tear prealbumin to be a member of the lipophilic-ligand carrier protein superfamily. J Biol Chem 267, 20282-20287 (1992).
17. P. Wojnar, M. Lechner, P. Merschak, B. Redl, Molecular cloning of a novel lipocalin-1 interacting human cell membrane receptor using phage display. J Biol Chem 276, 20206-20212 (2001).
18. P. Wojnar, M. Lechner, B. Redl, Antisense down-regulation of lipocalin-interacting membrane receptor expression inhibits cellular internalization of lipocalin-1 in human NT2 cells. J Biol Chem 278, 16209-16215 (2003).
19. M. Fluckinger, P. Merschak, M. Hermann, T. Haertle, B. Redl, Lipocalin-interacting-membrane-receptor (LIMR) mediates cellular internalization of beta-lactoglobulin. Biochim Biophys Acta 1778, 342-347 (2008).
20. Z. Zhang et al., Interaction of uteroglobin with lipocalin-1 receptor suppresses cancer cell motility and invasion. Gene 369, 66-71 (2006).
21. J. C. Christianson et al., Defining human ERAD networks through an integrative mapping strategy. Nat Cell Biol 14, 93-105 (2011).
22. V. S. Li et al., Wnt signaling through inhibition of beta-catenin degradation in an intact Axin1 complex. Cell 149, 1245-1256 (2012).
23. J. Seoane, H. V. Le, J. Massague, Myc suppression of the p21(Cip1) Cdk inhibitor influences the outcome of the p53 response to DNA damage. Nature 419, 729-734 (2002).
24. A. L. Haber et al., A single-cell survey of the small intestinal epithelium. Nature 551, 333-339 (2017).
25. S. J. Lloyd, S. Raychaudhuri, P. J. Espenshade, Subunit architecture of the Golgi Dsc E3 ligase required for sterol regulatory element-binding protein (SREBP) cleavage in fission yeast. J Biol Chem 288, 21043-21054 (2013).
26. X. Zeng et al., A dual-kinase mechanism for Wnt co-receptor phosphorylation and activation. Nature 438, 873-877 (2005).
27. P. Georgel, X. Du, K. Hoebe, B. Beutler, ENU mutagenesis in mice. Methods Mol Biol 415, 1-16 (2008).
28. A. S. Hidmark et al., Humoral responses against coimmunized protein antigen but not against alphavirus-encoded antigens require alpha/beta interferon signaling. J Virol 80, 7100-7110 (2006).
29. C. N. Arnold et al., A forward genetic screen reveals roles for Nfkbid, Zeb1, and Ruvb12 in humoral immunity. Proc Natl Acad Sci USA 109, 12286-12293 (2012).
30. J. H. Choi et al., A single sublingual dose of an adenovirus-based vaccine protects against lethal Ebola challenge in mice and guinea pigs. Mol Pharm 9, 156-167 (2012).
31. M. Oukka, M. Cohen-Tannoudji, Y. Tanaka, C. Babinet, K. Kosmatopoulos, Medullary thymic epithelial cells induce tolerance to intracellular proteins. J Immunol 156, 968-975 (1996).
32. K. Crozat et al., Analysis of the MCMV resistome by ENU mutagenesis. Mamm Genome 17, 398-406 (2006).
33. Z. Zhang et al., Insulin resistance and diabetes caused by genetic or diet-induced KBTBD2 deficiency in mice. Proc Natl Acad Sci USA 113, E6418-E6426 (2016).

### Example 2: Generation of LMBR1L Monoclonal Antibody

Based on analysis of the NCBI Mus musculus (www.ncbi.nlm.nih.gov/gene/74775) Lmbr1l and Homo sapiens (www.ncbi.nlm.nih.gov/gene/55716) (FIG. 2) LMBR1L EST libraries determined that three (NP_083374.1, XP_011244060.1, and XP_017172262.1) and fifteen (NP_060583.2, NP_001287679.1, NP_001287680.1, NP_001339090.1, NP_001339091.1, NP_001339092.1, NP_001339093.1, NP_001339094.1, NP_001339096.1, XP_016875117, XP_016875118, XP_016875116, XP_016875115, XP_016875120, and XP_011536866) fragments were predicted as alternatively spliced transcripts, respectively. Human NP_060583.2 and murine NP_083374.1 as canonical LMBR1L proteins are 489-residue long with 9 transmembrane domains. Human LMBR1L protein has 97% identity to the murine LMBR1L protein (FIG. 20). The five extracellular domains of canonical human LMBR1L are marked in FIG. 1B. Based on this analysis, there are five targetable extracellular peptide sequences that are candidates for the anti-human LMBR1L inhibitors such as LMBR1L antibodies.

A portion of any one or more of the five extracellular domains (amino acids 1-21, 88-114, 176-196, 327-350 and 410-431, respectively, see FIG. 1B), as opposed to the full length, can also be used as an immunogen. Different methods known in the art, and those that have been disclosed herein, may be used to generate monoclonal, fully human or humanized anti-LMBR1L antibodies. For example, as described above, fully human LMBR1L antibodies can also be produced from phage-display libraries. Humanized anti-LMBR1L antibodies can be prepared by humanizing monoclonal antibodies obtained from hybridomas.

An exemplary approach can include:
1. Use phage display to identify binding antibodies reactive with extracellular loops of the LMBR1L protein, displayed by expressing the protein on liposomes.
2. Upon finding such binding antibodies, re-screen for inhibition of LMBR1L activity using human lymphoid cells. The inhibition of activity would be detected by measuring a rise in nuclear β-catenin and c-Myc in cells following addition of the antibody.
3. Optimize affinity and engineer into antibody Fab or IgG molecule for production.

The LMBR1L protein is strongly conserved between humans and mice (FIG. 20). Use of phage display is likely to produce a reagent reactive with both species, useful in preclinical and clinical testing.

In another example, a C-terminal His tag, suitable for purification by affinity chromatography, can be added to the immunogen. Purified protein can be inoculated into mice together with a suitable adjuvant. Monoclonal antibodies produced in hybridomas can be tested for binding to the immunogen, and positive binders can be screened (e.g., decreased T cell-dependent and T-cell independent antibody responses, decreased T cells, B cells, NK cells and NK T cells) for ability to affect β-catenin, FRIZZLED-6, ZNRF3, and/or c-Myc expression in human lymphoid cells in the assays described above. Thereafter, antibodies can be humanized for preclinical and clinical studies.

As a cell surface molecule, LMBR1L should be accessible to inhibition by an antibody. This would be reasonably expected to mimic the effects of the mutation. An antibody inhibitor of LMBR1L could be used to arrest, for example, graft-versus-host disease, allograft rejection, or autoimmune diseases, including (but not limited to) systemic lupus erythematosus, Hashimoto's thyroiditis, Grave's disease, type I diabetes, multiple sclerosis, and rheumatoid arthritis.

It would also be reasonable to expect that administration of such an antibody would be effective after disease has developed, since dissociation of the Wnt receptor from the release of active β-catenin to the nucleus would bring about the rapid death of all activated lymphoid cells including T cells, B cells, NK and NK T cells (less so non-activated cells) via programmed cell death. While some developmental requirements for LMBR1L may exist since fewer than the expected number of homozygotes are observed at weaning age, we do not know of abnormalities in mature homozygous knockout mice other than immune abnormalities, suggesting that this component of the Wnt signaling pathway is probably immune-specific in its action, at least post-developmentally. An antibody against LMBR1L would be more selective than chemical cytoreductive reagents such as cyclophosphamide, or antibodies such as anti-lymphocyte globulin.

### OTHER EMBODIMENTS

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and patent applications referenced in this specification are incorporated herein by reference in their entirety for all purposes to the same extent as if each individual publication, patent or patent application were specifically indicated to be so incorporated by reference.

1. An antagonist of limb region 1 like (LMBR1L) for use in the treatment of a condition associated with an excessive or overactive immune system, wherein preferably the condition is selected from an inflammatory disease, autoimmune disease, graft versus host disease, or an allograft rejection.

2. A method of identifying an antagonist useful in the treatment of a condition associated with an excessive or overactive immune system, the method comprising determining the binding of a test compound to LMBR1L, and determining an activity of LMBR1L is reduced by the test compound compared to a control, wherein preferably the condition is selected from an inflammatory disease, autoimmune disease, graft versus host disease, or an allograft rejection.

3. A method of identifying an individual with of a condition associated with an excessive or overactive immune system that is suitable for treatment with an LMBR1L antagonist, comprising determining an activity or amount of LMBR1L in a sample obtained from the individual, wherein an increased activity or amount compared to a control indicates that the individual is suitable for treatment with an LMBR1L antagonist, wherein preferably the condition is selected from an inflammatory disease, autoimmune disease, graft versus host disease, or an allograft rejection.

4. A method of providing an immune suppressive therapy, comprising inhibiting limb region 1 like (LMBR1L) in a subject in need thereof, thereby suppressing an immune response.

5. The method of claim 4, wherein said inhibiting comprises reducing the number of common lymphoid progenitors and/or lymphocytes in the subject.

6. The method of claim 5, wherein the lymphocytes comprise one or more of T cells, B cells, NK and NK T cells.

7. The method of claim 4, wherein the subject has an inflammatory disease, autoimmune disease, graft versus host disease, or an allograft rejection.

8. The method of claim 7, wherein the autoimmune disease is systematic lupus erythematosus (SLE), Hashimoto's thyroiditis, Grave's disease, type I diabetes, multiple sclerosis and/or rheumatoid arthritis.

9. The method of claim 4, comprising administering to the subject an effective amount of an LMBR1L inhibitor, wherein the LMBR1L inhibitor binds to LMBR1L, preferably an extracellular domain of LMBR1L.

10. A composition for treating an immunodeficiency disorder, comprising a nucleic acid encoding LMBR1L into a subject in need thereof.

11. A method for treating an immunodeficiency disorder, comprising introducing a nucleic acid encoding LMBR1L into a subject in need thereof.

12. A method of reducing lymphopoiesis in a subject having a condition associated with an excessive or overactive immune system, comprising administering to the subject a therapeutically effective amount of an LMBR1L antagonist.
